(19) United States
(12) Patent Application Publication
(10) Pub. No.: US 2024/0287532 A1
(43) Pub. Date: Aug. 29, 2024

(54) RIBOSWITCH MODULES AND METHODS FOR CONTROLLING PROTEIN EXPRESSION IN PLANTS

(71) Applicants: BASF Corporation, Florham Park, NJ (US); President and Fellows of Harvard College, Cambridge, MA (US)

(72) Inventors: Yanshun LIU, Research Triangle Park, NC (US); Brody John DEYOUNG, Research Triangle Park, NC (US); Shirong ZHANG, Research Triangle Park, NC (US); Laura SCHOUTEN, Research Triangle Park, NC (US); Peifeng REN, Research Triangle Park, NC (US); Joerg BAUER, Research Triangle Park, NC (US); Evan M. ZHAO, Boston, MA (US); James J. COLLINS, Boston, MA (US); Xiao TAN, Boston, MA (US); Fei RAN, Boston, MA (US); Angelo S. MAO, Somerville, MA (US); Helena DE PUIG GUIXE, Gaia Barcelona (ES); Emma J. CHORY, Boston, MA (US)

(21) Appl. No.: 18/008,935

(22) PCT Filed: Jun. 11, 2021
(86) PCT No.: PCT/US2021/037077

§ 371 Date: Dec. 7, 2022

Related U.S. Application Data
(60) Provisional application No. 63/038,536, filed on Jun. 12, 2020.

(51) Int. Cl. C12N 15/82; C12N 5/04

(52) U.S. Cl. CPC C12N 15/8203; C12N 5/04; C12N 15/8279; C12N 2770/32043

(57) ABSTRACT

The present disclosure provides genetic constructs comprising a recombinant internal ribosome entry site (IRES), which may be used as riboswitches to modulate translation of an operably-mRNA sequence encoding a protein of interest in a plant. In other aspects, the disclosure provides recombinant plant cells, methods, kits and systems that utilize the same, e.g., to provide a platform for modulating the expression of essentially any protein of interest in a plant cell.

### CROSS-REFERENCE TO RELATED APPLICATION

This application claims priority to U.S. Provisional Application No. 63/038,536, filed on Jun. 12, 2020, the entire contents of which is hereby incorporated by reference.

### TECHNICAL FIELD

The disclosure provides constructs and methods for modulating protein expression in plant cells using recombinant Group 1 internal ribosome entry site (IRES) elements derived from viral IRES elements.

### PARTIES TO A JOINT RESEARCH AGREEMENT

The claimed invention was made by, on behalf of, and/or in connection with one or more of the following parties to a joint research agreement: President and Fellows of Harvard College and BASF Corporation. The joint research agreement was in effect on and before the effective filing date of the claimed invention, and the claimed invention was made as a result of activities undertaken within the scope of the joint research agreement.

### BACKGROUND OF THE DISCLOSURE

The regulation of gene expression is critical for growth and development, as well as for the proper maintenance of homeostasis in the face of changing environmental conditions. As such, cells utilize a variety of mechanisms to increase or decrease the production of specific gene products (e.g., proteins or RNA). Expression levels may be modulated, e.g., to trigger developmental pathways, in response to environmental stimuli, or to adapt to new food sources. Gene expression may be modulated at the transcriptional level, e.g., by increasing or decreasing the rate of transcriptional initiation, or aspects of RNA processing. It may also be controlled the post-translational modification of proteins (e.g., by increasing or decreasing the rate of degradation). A myriad of different mechanisms for controlling gene expression exist in nature, and these mechanisms are typically linked to form complex regulatory networks. The use of different mechanisms and triggers permits cells to express specific subsets of genes, or to adjust the level of particular gene products, on an as-needed basis. Doing so conserves energy and resources while also allowing cells to respond more quickly to environmental stimuli. For example, bacteria and eukaryotic cells often adjust the expression of enzyme used in synthetic or metabolic pathways based upon the availability of required substrates or end products. Similarly, many cell types will induce synthesis of protective molecules (e.g., heat shock proteins) in response to environmental stress.

A number of approaches have been developed in order to artificially control levels of gene expression, many of which are modeled on naturally occurring regulatory systems. In general, gene expression can be controlled at the level of RNA transcription or post-transcriptionally, e.g., by controlling the processing or degradation of mRNA molecules, or by controlling their translation. For example, gene expression may be modulated by the administration of small molecule activators or inhibitors (e.g., to increase or decrease the activity of transcription factors), or by the administration of nucleic acids designed to inactivate or degrade mRNA (e.g., using ribozymes, antisense DNA/RNA, and RNA interference techniques). Although these approaches have proven to be useful in many applications, their usefulness may be limited by certain drawbacks. For example, ribozyme, antisense DNA/RNA, and RNAi-based methods normally require a sequence-specific approach (e.g., the small-interfering RNAs used for RNAi and antisense DNA/RNA must be specifically designed for each target). Moreover, the use of small molecule activators and inhibitors to modulate transcription is also non-ideal because such methods typically have a slow response time.

Research efforts to address these shortcomings have led to the development of prokaryotic RNA-sensing modules, referred to as “toehold switches,” which rely on trigger-based unfolding of a ribosome binding site (RBS). See, for example, U.S. Pat. No. 10,208,312, the entire contents of which is hereby incorporated by reference. Toehold switches selectively repress translation of a target transcript by hiding the RBS in the absence of a separate trigger RNA (“trRNA”) and reveal the RBS in the presence of the trRNA, resulting in the initiation of translation of an operably-linked sequence encoding a protein of interest. Prokaryotic toehold switches partially address the shortcoming of other prior art methods by providing an efficient mechanism for modulating translation in prokaryotic organisms. However, this toehold switch mechanism is generally incompatible with eukaryotic systems, which rely on a more complicated set of epigenetic signals to initiate and regulate translation.

### BRIEF SUMMARY OF EXEMPLARY EMBODIMENTS OF THE DISCLOSURE

The present disclosure addresses various needs in the art by providing new genetic constructs and methods for modulation protein translation. These constructs, for example, can be used as a platform to regulate the translation of arbitrary proteins of interest in plant cells without the need for sequence-specific design modifications. Moreover, the systems described herein allow for the artificial control of gene expression within plant cells in response to external stimuli.

In particular, the present disclosure describes genetic constructs, recombinant cells, methods, kits and systems that, for example, provide a platform for modulating the expression of essentially any protein of interest in a plant cell.

In a first general aspect, the present disclosure provides recombinant IRES modules engineered to reduce or prevent translation of an operably-linked mRNA sequence encoding a protein of interest. These recombinant IRES modules are further engineered to fold into an activated form in the presence of a specific trRNA. Once activated, translation of the operably-linked mRNA sequence is allowed to proceed. The trRNA can be an artificial sequence introduced into the plant cell (e.g., by a plasmid or chemically-mediated transfection) or a sequence found in a naturally-occurring mRNA (e.g., a viral mRNA). As such, these recombinant IRES modules can be used to modulate translation of a protein of interest for therapeutic, agricultural or industrial applications, and can also be used as a sensor to detect exogenous stimuli, such as a viral infection.

In another general aspect, the disclosure provides, for use in a plant or plant cell, a recombinant nucleic acid molecule, comprising: a) a first segment encoding a Group 1 Dicistroviridae internal ribosome entry site (IRES) that has been modified to incorporate exogenous nucleotide sequences at a first site and a second site, and b) a second segment encoding a protein, downstream from and operably linked to the first segment such that translation of the protein is repressed when the IRES is in an inactivated state; wherein the first site comprises a first nucleotide sequence, and the second site comprises a second nucleotide sequence which is the reverse complement of at least a portion of the first nucleotide sequence. In some aspects, the nucleic acid molecule is an mRNA. In some aspects, the second nucleotide sequence is the reverse complement of substantially all of the first nucleotide sequence. In some aspects, the Group 1 Dicistroviridae IRES is a cricket paralysis virus (CrPV), Kashmir bee virus (KBV), acute bee paralysis virus (ABPV), or Plautia stali intestine virus (PsIV) IRES.

In some aspects, the Group 1 Dicistroviridae IRES has been modified to incorporate exogenous nucleotide sequences at a first site and a second site, wherein the first and second sites are each independently selected from any of Site 1, Site 2, Site 3, Site 4, Site 5, Site 6, Site 7, and Site 8 (Sites 1-8 are defined below and shown in the schematic provided as FIG. 2). In some aspects, the first and second sites respectively comprise: Site 1 and Site 2, Site 1 and Site 4, Site 1 and Site 5, Site 1 and Site 6, Site 1 and Site 7, Site 1 and Site 8, Site 2 and Site 6, Site 2 and Site 7, Site 4 and Site 6, Site 5 and Site 6, Site 5 and Site 7, Site 6 and Site 7, Site 8 and Site 2, Site 8 and Site 6, or Site 8 and Site 7.

In some aspects, the first nucleotide sequence is 25-80 nt in length. In other aspects, the first nucleotide sequence may have a length within a subrange (e.g., a length of 30-40 nt, 40-50 nt, 50-60 nt, or a length within a subrange defined by any pair of integer values within the range of 25-80 nt). In some aspects, the second nucleotide sequence is 8-25 nt in length. In other aspects, the second nucleotide sequence may have a length within a subrange (e.g., a length of 10-15 nt, 15-25 nt, or a length within a subrange defined by any pair of integer values within the range of 8-25 nt).

In some aspects, the first and second nucleotide sequences are capable of hybridizing when expressed in a plant cell under in vivo or in vitro conditions, causing the Group 1 Dicistroviridae IRES to fold into an inactivated state.

In still further aspects, the Group 1 Dicistroviridae IRES is configured to fold into an activated state in the presence of a trigger RNA molecule comprising a third nucleotide sequence, wherein the third nucleotide sequence is the reverse compliment of the first nucleotide sequence. The first nucleotide sequence may be capable of hybridizing to the third nucleotide sequence when expressed in a eukaryotic cell under in vivo or in vitro conditions, causing the Group 1 Dicistroviridae IRES to fold into the activated state

In another general aspect, the disclosure provides plasmids and plant cells encoding any of the recombinant nucleic acid molecules (e.g., any recombinant IRES) described herein. With respect to the plant cells, it is contemplated that the such recombinant nucleic acid molecules may be incorporated into the genomic or plasmid DNA of the plant cell.

In another general aspect, the disclosure provides systems and kits that may be used to modulate gene expression in a plant cell. For instance, the disclosure provides a system for the control of gene expression, comprising: a) a recombinant nucleic acid molecule according to any aspect described herein; and b) a trigger RNA molecule comprising a third nucleotide sequence, wherein the third nucleotide sequence is the reverse compliment of the first nucleotide sequence of the recombinant nucleic acid molecule. Similarly, the disclosure provides kits comprising: a) a plasmid encoding any of the recombinant nucleic acid molecules described herein; and b) a trigger RNA molecule comprising a third nucleotide sequence, wherein the third nucleotide sequence is the reverse compliment of the first nucleotide sequence of the recombinant nucleic acid molecule

In another general aspect, the disclosure provides, for use in plants or plant cells, a recombinant mRNA molecule, comprising: a) a first segment encoding a first protein; b) a second segment, downstream of the first segment, encoding a Group 1 Dicistroviridae IRES that has been modified to incorporate exogenous nucleotide sequences at a first site and a second site; and c) a third segment encoding a second protein, downstream from and operably linked to the second segment such that translation of the second protein is repressed when the IRES is in an inactivated state; wherein transcription of the recombinant mRNA molecule is dependent on RNA Polymerase II, and wherein the first site comprises a first nucleotide sequence, and the second site comprises a second nucleotide sequence which is the reverse complement of at least a portion of the first nucleotide sequence. Such constructs may display reduced translational leakiness compared to other constructs described herein.

In another general aspect, the disclosure provides methods of using the recombinant nucleic acid molecules (e.g., recombinant IRES elements) described herein, in various applications. For example, a method of activating and/or modulating expression of a protein, may comprise: a) providing a plant cell engineered to express any of the recombinant nucleic acid molecules described herein; b) introducing a trigger RNA molecule comprising a third nucleotide sequence into the plant cell, wherein the third nucleotide sequence is the reverse compliment of the first nucleotide sequence of the recombinant nucleic acid molecule; wherein the first nucleotide sequence hybridizes to the third nucleotide sequence under in vivo conditions, causing the Group 1 Dicistroviridae IRES to fold into an activated state. In some aspects, the plant cell engineered to express the recombinant nucleic acid molecule is provided by introducing any of the recombinant nucleic acid molecules described herein into the plant cell.

As noted above, the recombinant IRES elements described herein may be used as sensors to detect external stimuli. Accordingly, the disclosure provides methods for detecting bacterial or viral infection of a plant cell, comprising: a) providing a plant cell engineered to express one of the recombinant nucleic acid molecules described herein, wherein the first nucleotide sequence of the recombinant nucleic acid molecule is configured to be the reverse compliment of at least a portion of a mRNA sequence unique to a bacterium or virus; and b) determining whether the plant cell is infected with the bacterium or virus by detecting and/or measuring the presence of the protein encoded by the second segment of the recombinant nucleic acid molecule.

In still further aspects, the disclosure provides methods for controlling differentiation of a plant cell, comprising a) providing a plant cell engineered to express any of the recombinant nucleic acid molecules described herein; and b) culturing the plant cell; wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of the selected cell type.

Various exemplary aspects of the presently disclosed inventions are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 is a diagram summarizing traditional IRES-mediated eukaryotic gene expression using an unmodified IRES.

FIG. 2 is a schematic representation of a Group 1 CrPV IRES, highlighting the three major Loops (or domains) of this IRES. The architecture of Group 1 IRES elements is conserved among Dicistroviridae family members (e.g., CrPV, KBV, and ABPV).

FIG. 3 is a schematic representation of a Group 1 CrPV IRES, highlighting 8 sites (i.e., “Site 1,” “Site 2,” . . . “Site 8”), which can be used as insertion regions for the exogenous nucleic acid sequences described herein.

FIG. 4 is a diagram summarizing eukaryotic gene expression using an exemplary recombinant IRES described herein.

FIG. 5 is a schematic representation of an mRNA construct encoding one of the recombinant IRES elements described herein, as well as a second upstream gene.

FIG. 6 is a graph showing the activity level of different IRES modules. The series, from left to right in FIG. 6 are “+T7 pol+GFP (Trigger)”, “+T7pol−GFP (Trigger)”, “−T7pol+GFP (Trigger)”, and “−T7pol−GFP (Trigger).”

FIG. 7 is a graph showing the results of a screen of recombinant IRES riboswitch constructs with a pair of exogenous nucleotide sequence introduced at various sites. The series, from left to right in FIG. 7 are “+T7 pol+GFP (Trigger)”, “+T7pol−GFP (Trigger)”, “−T7pol+GFP (Trigger)”, and “−T7pol−GFP (Trigger).”

FIG. 8 is a graph showing the effect of choosing exogenous nucleotide sequences with matching base pairs that break the specified fold and pseudoknot regions. The series, from left to right in FIG. 8 are “+T7 pol+GFP (Trigger)”, “+T7pol−GFP (Trigger)”, “−T7pol+GFP (Trigger)”, and “−T7pol−GFP (Trigger).”

FIG. 9 is a graph showing the effect of switching promoters and adding an upstream activation sequence for RNA polymerase I. The series, from left to right in FIG. 10 are “GFP Trigger”, “Azurite Trigger”, and “ySUMO Trigger.”

FIG. 10 is a graph showing that exemplary recombinant IRES riboswitches described herein are highly specific for their respective trigger RNAs (trRNAs).

FIG. 11 is a graph showing the effect of mutations on the functionality of exemplary recombinant IRES riboswitches described herein.

FIG. 12 is a graph showing that recombinant IRES riboswitches may be based on the sequences of IRES modules produced by several Dicistroviridae members (e.g., KBV and ABPV).

FIG. 13 is a diagram showing the use of a recombinant IRES according to the disclosure in a eukaryotic cell as a sensor to detect a viral infection.

FIG. 14 is a graph showing the results of a luciferase assay which was used to evaluate several exemplary IRES riboswitches that were transiently expressed in tobacco plants. The data points shown in this graph represent average values for each set of replicates.

FIG. 15 is a graph showing the raw data obtained from the same luciferase assay that was used to generate FIG. 14.

### DETAILED DESCRIPTION

There exists a need in the art for new constructs that can be used as a platform to regulate the translation of arbitrary proteins of interest in plant cells without the need for sequence-specific design modifications. Traditional options (e.g., ribozyme, antisense DNA/RNA, and RNAi-based methods) normally require a sequence-specific approach, potentially limiting their usefulness. More recent developments such as prokaryotic toehold switches address some of the shortcomings of traditional options. However, their usefulness is generally confined to prokaryotic organisms due to differences between the translation mechanisms used by prokaryotes and eukaryotes. For example, eukaryotic translation initiation relies on endogenous RNA polymerase II-recruited 5′ modified capping, a poly-adenosine (polyA) tail for mRNA stabilization, and a kozak sequence for protein translational regulation. Although the kozak sequence improves ribosomal binding, it is not an ideal RBS substitute; previously developed kozak-based toeholds have only achieved a maximum two-fold trRNA-driven induction of translation. As such, toehold switches compatible with eukaryotic cells provide limited utility at this time.

In recent years, more complex RNA-based switches have been developed utilizing Cas9 expression and folding of the guide RNA (gRNA). Unfolding of the gRNA leads to activation of the Cas9 enzyme and corresponding repression or activation. Yet, despite bulky circuitry, these mechanisms also induce only modest fold changes (in both eukaryotes and prokaryotes). Similarly, recent advancements in the area of ribozyme research have led to the development of ribozymes that cleave the polyA tail of a target mRNA upon small molecule induction. However, ribozyme-based mechanisms are currently limited to small-length trRNAs, limiting their ability to be tuned for specific sequences, and in any event are limited exclusively to an “ON-to-OFF” sensor, which is non-ideal for leakiness and induction tuning.

The present disclosure addresses these and other shortcomings of known mechanisms for regulating eukaryotic gene expression by providing a minimal component RNA-based sensor (i.e., the recombinant IRES element described herein) that responds “OFF-to-ON” in response to a specific trRNA with a high “ON” vs “OFF” fold change. As such, these constructs are advantageous in expression systems used to produce proteins for industrial, agricultural, or therapeutic use, as well as in other novel applications (e.g., in biosensors capable of detecting environmental stimuli such as the presence of viral mRNA).

### Eukaryotic and Viral Translation Mechanisms

In eukaryotes, protein translation is normally initiated by a tightly-regulated mechanism that requires a modified nucleotide ‘cap’ on the 5′ end of a mRNA, as well as initiation factor proteins (eIFs) that recruit and position the ribosome. In order to bypass this system, many pathogenic viruses use an alternative, cap-independent mechanism that relies upon the use of specific RNA secondary (or tertiary) structures to recruit and manipulate the ribosome, as a substitute for the 5′ cap and eIFs used during the canonical pathway. The RNA elements driving this process are known as IRESs.

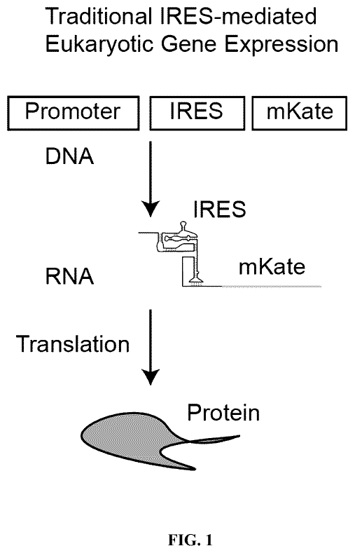

FIG. 1 illustrates the process by which an unmodified viral IRES can be used to express of an arbitrary protein (in this case, mKate). In brief, traditional IRES-mediated eukaryotic gene expression requires a promoter (e.g., a T7 promoter) operably-linked to a downstream DNA segment encoding a viral IRES and a gene of interest. In this example, the T7 promoter recruits T7 RNA polymerase (which does not 5′ cap mRNA) to transcribe an mRNA comprising the IRES and a segment encoding the protein of interest, i.e., mKate. The viral IRES will normally recruit a ribosome (and potentially other components necessary for translation), resulting in expression of the mKate protein. In this example, the IRES is an unmodified viral IRES rather than a recombinant IRES according to the disclosure, which would be inactive in this example due to the absence of a trRNA.

Viral IRESs have been organized into four distinct groups based on the secondary and tertiary structures of their RNA elements and their mode of action for initiating translation. Within this classification system, Group 1 IRESs are generally more compact and more complex than IRESs in Groups 2-4. Moreover, Group 1 IRESs are notable because they can initiate translation on a non-AUG start codon, do not require any eIFs and do not use the initiator Met-tRNA. Group 1 IRESs are consequently able to promote efficient translation initiation, requiring only the small and large ribosomal subunits. Several Dicistroviridae family members (e.g., cricket paralysis virus (CrPV), Kashmir bee virus (KBV), and acute bee paralysis virus (ABPV)) are known to encode Group 1 IRESs. Group 1 IRESs are highly conserved in terms of sequences, secondary and tertiary structures among Dicistroviridae family members. The CrPV IRES is the most well-studied IRES in this group and is representative of other Group 1 Dicistroviridae IRESs (e.g., of the KBV and ABPV IRESs).

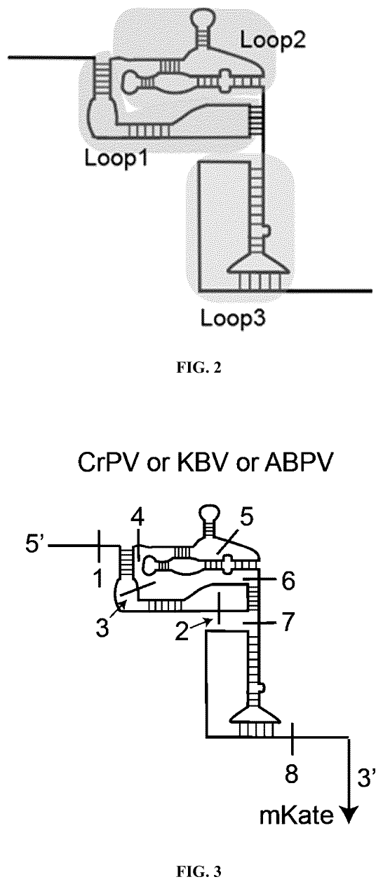

FIG. 2 shows a schematic representation of the secondary structure of the CrPV Group 1 IRES. As illustrated by FIG. 2, the CrPV Group 1 IRES normally folds into a compact structure which has three major loops (or domains) labeled here as Loops 1-3, each including a pseudoknot structure (referred to as PKI, PKII, and PKIII, respectively), as well as internal loops, bulges, and hairpin motifs. This folded structure is essential for IRES activity. For example, the triple-pseudoknot architecture is known to functionally substitute for the initiator met-tRNA during internal initiation, directing translation initiation at a non-AUG triplelet. Normally, the presence of the CrPV Group 1 IRES on a viral mRNA would recruit a eukaryotic ribosome to the mRNA and initiate the translation of the encoded viral protein.

### Recombinant IRES Riboswitches

In some aspects, the present disclosure relates to nucleic acid constructs (e.g., mRNA) which have been modified to incorporate at least one recombinant IRES riboswitch. Embodiments of these recombinant IRES riboswitches can be referred to herein as “eToeholds.” The recombinant IRES riboswitch can be derived from, or comprises sequences naturally-occurring in a viral IRES. The recombinant IRES can be a viral IRES modified to comprise an exogenous, e.g, non-endogenous sequence. In some aspects, the recombinant viral IRES comprises a viral IRES comprising two insertions of exogenous, e.g., non-endogenous sequences. In some aspects, an insertion in a viral IRES to create a recombinant viral IRES riboswitch described herein can comprise deletion of viral sequences at the insertion site(s). In some aspects, an insertion in a viral IRES to create a recombinant viral IRES riboswitch described herein does not comprise deletion of viral sequences at the insertion site(s).

IRES riboswitches described herein can be derived from any IRES sequence obtained or naturally-occurring in a viral genome or sequence. In some aspects, the IRES riboswitches described herein can be derived from any IRES sequence obtained or naturally-occurring in a eukaryotic (e.g., plant) pathogenic or plant (e.g., plant) commensal viral genome or sequence. Such viruses and their sequences are known in the art. In some aspects, the IRES sequence can be a Group 1 IRES. In some aspects, the IRES sequence can be a Group 1, Group 2, Group 3, or Group 4 IRES. In some aspects, the IRES is derived from an IRES sequence of, or the IRES that is modified is an IRES sequence of a Group 1 Discistroviridae IRES; a Hepacivirus IRES; or an Enterovirus IRES. Exemplary wild-type IRES sequences and recombinant IRES riboswitch sequences are provided herein and further wild-type IRES sequences for use in the methods and compositions described herein are readily obtained and/or identified by one of ordinary skill in the art. For example, a database of IRES sequences is available on the world wide web at iresite.org.

In some aspects, the Hepacivirus IRES is derived from an IRES sequence of, or the IRES that is modified is an IRES sequence of a hepatitis C virus (HCV); a hepatitis B virus; a hepatitis F virus; a hepatitis I virus; a hepatitis J virus; a hepatitis K virus; a hepatitis L virus; a hepatitis M virus; a hepatitis N virus; a Guereza hepacivirus; a hepatitis GB virus B virus; a non-primate hepacivirus NZP1 virus; a Norway rate hepacivirus 1 virus; a Norway rate hepacivirus 2 virus; a bat hepacivirus; a bovine hepacivirus; an equine hepacivirus; a hepacivirus P virus; a rodent hepacivirus; and a wenling shark virus. In some aspects, the Hepacivirus IRES is derived from an IRES sequence of, or the IRES that is modified is an IRES sequence of a hepatitis c virus (HCV). Sequences for such viruses are known in the art, e.g., they are available on the world wide web at ncbi.nlm.nih.gov/genomes/GenomesGroup.cgi?taxid=11102.

In some aspects, the Enterovirus IRES is derived from an IRES sequence of, or the IRES that is modified is an IRES sequence of a poliovirus (PV); enterovirus 71 (EV71); Enterovirus A virus (e.g., coxsackievirus A2; enterovirus A; or enterovirus A114); Enterovirus B virus (e.g., coxsackievirus B3 or enterovirus B); Enterovirus C; Dromedary camel enterovirus 19CC; Enterovirus D virus (e.g., Enterovirus D or Enterovirus D68); Enterovirus E; Enterovirus F virus (e.g., Enterovirus F or possum enterovirus W1); Enterovirus H virus (e.g., Enterovirus H or simian enterovirus SV4); Enterovirus J virus; Enterovirus SEV-gx; Rhinovirus A virus (e.g., human rhinovirus A1 or rhinovirus A); Rhivnovirus B virus (e.g., human rhinovirus B2 or rhinovirus B14); Rhinovirus C virus (e.g., human rhinovirus NAT001 or rhinovirus C); picornaviridae virus (e.g., picornaviridae sp. Rodent/Ee/PicoV/NX2015); porcine enterovirus (e.g., porcine enterovirus 9); Enterovirus AN12; Enterovirus goat/JL14; Sichuan takin enterovirus; or Yak enterovirus. In some aspects, the Enterovirus IRES is derived from an IRES sequence of, or the IRES that is modified is an IRES sequence of a poliovirus (PV) or enterovirus 71 (EV71). Sequences for such viruses are known in the art, e.g., they are available on the world wide web at ncbi.nlm.nih.gov/genomes/GenomesGroup.cgi?taxid=12059.

In some aspects, the IRES riboswitches described herein are derived from Group I IRES elements used by members of the Dicistroviridae family of viruses (e.g., CrPV, KBV, or ABPV). In some aspects, the Group I Discistroviridae IRES is derived from an IRES sequence of, or the IRES that is modified is an IRES sequence of a cricket paralysis virus (CrPV), a Kashmir bee virus (KBV), an acute bee paralysis virus (ABPV), a Plauta stali Intestine Virus (PSIV) IRES; an aphid lethal paralysis virus (ALPV) IRES; a black queen cell virus (BQCV) IRES; a Drosophila C virus (DCV) IRES; a Himetobi P virus (HiPV) IRES; a Homalodisca coagulata virus-1 (HoCV-1) IRES; a Rhopalosiphum padi virus (RhPV) IRES; or a Triatoma virus (TrV).

As explained above, the naturally-occurring form of these IRES elements recruits ribosomes to an associated mRNA, resulting in translation of the mRNA (i.e., a regulatory circuit that is constitutively active). The co-inventors of the present invention have surprisingly found that Dicistroviridae IRES elements may be genetically modified to produce a recombinant IRES riboswitch that can be switched “ON” or “OFF” based upon the concentration of separate trigger RNA (trRNA) molecule. This new functionality is provided by inserting two or more segments comprising exogenous nucleotide sequences into the original sequence of a viral IRES element. As explained in further detail below, these segments are designed to hybridize in the absence of a corresponding trRNA, causing the recombinant IRES to fold into an inactive state. When the trRNA is provided, hybridization between the two segments is disrupted, allowing the recombinant IRES to fold into a conformation similar to that of the naturally-occurring viral IRESs, which are constitutively active as noted above. The recombinant IRES consequently functions as a riboswitch that can be switched “ON” or “OFF” based upon the concentration of the corresponding trigger RNA, modulating the translation of an operably-linked downstream mRNA sequence encoding a protein of interest.

In some aspects, the IRES riboswitches described herein comprise a nucleotide sequence that shares at least 70% sequence identity with a viral IRES (e.g., a Hepacivirus IRES; or an Enterovirus IRES). In some aspects, the IRES riboswitches described herein display at least 90, 95, 98, 99 or 100% sequence identity with a viral IRES (e.g., a Hepacivirus IRES; or an Enterovirus IRES) at all positions except for the two segments comprising exogenous nucleotide sequences. In some aspects, an IRES riboswitch according to the disclosure comprises a nucleotide sequence that shares at least 70, 80, 85, 90, 95, 98, 99 or 100% sequence identity to that of any one of SEQ ID NOs:11-17, except for the presence of exogenous nucleotide sequences inserted at two sites within this sequence, which are indicated by an X in the sequences. In some aspects, an IRES riboswitch according to the disclosure comprises a nucleotide sequence with at least 80% sequence identity to any one of SEQ ID NOs:11-17, except for the presence of exogenous nucleotide sequences inserted at two sites within this sequence, which are indicated by an X in the sequences. In some aspects, an IRES riboswitch according to the disclosure comprises a nucleotide sequence with at least 85% sequence identity to any one of SEQ ID NOs: 11-17, except for the presence of exogenous nucleotide sequences inserted at two sites within this sequence, which are indicated by an X in the sequences. In some aspects, an IRES riboswitch according to the disclosure comprises a nucleotide sequence with at least 95% sequence identity to any one of SEQ ID NOs: 11-17, except for the presence of exogenous nucleotide sequences inserted at two sites within this sequence, which are indicated by an X in the sequences. The exogenous nucleotide sequences inserted at the two sites can be first and second nucleotide sequences as described elsewhere herein.

In some aspects, the IRES riboswitches described herein comprise a nucleotide sequence that shares at least 70% sequence identity with a Group I Dicistroviridae IRES (e.g., a CrPV, KBV, or ABPV IRES. In some aspects, the IRES riboswitches described herein display at least 90, 95, 98, 99 or 100% sequence identity with a Group I Dicistroviridae IRES at all positions except for the two segments comprising exogenous nucleotide sequences. For example, an IRES riboswitch according to the disclosure may comprise a nucleotide sequence that shares at least 90, 95, 98, 99 or 100% sequence identity to that of SEQ ID NO:1, except for the presence of exogenous nucleotide sequences inserted at two sites within this sequence.

In some aspects, the recombinant IRES is derived from an IRES sequence of, or the IRES that is modified is an IRES sequence of a virus other than coxsackievirus B3 (CVB3). In some aspects, the recombinant IRES is not derived from an IRES sequence of, or the IRES that is modified is not an IRES sequence of coxsackievirus B3 (CVB3). In some aspects, the recombinant IRES is derived from an IRES sequence of, or the IRES that is modified is an IRES sequence of an Enterovirus other than coxsackievirus B3 (CVB3).

In the following explanations of the structure and function of the present recombinant IRES riboswitch technology, reference is made to figures depicting Group 1 Dicistroviridae IRESs and recombinant IRES riboswtiches derived therefrom. These figures are illustrative of exemplary aspects and do not imply that the technology is limited to these aspects.

FIG. 3 shows a schematic representation of the CrPV Group 1 IRES, annotated with numeric labels identifying 8 potential insertion sites (Sites 1-8). As noted above, this structure is representative of the structures of other Group 1 Dicistroviridae IRESs (e.g., the KBV and ABPV Group 1 IRESs). These sites shall be referenced herein in various aspects of the disclosure. For example, a recombinant IRES according to the disclosure may comprise an IRES which has a secondary structure that is identical or substantially similar to the secondary structure shown in FIG. 3, but which includes at least one exogenous RNA segment inserted at one or more of Sites 1-8. For example, a recombinant IRES may comprise a sequence derived from the CrPV, KBV, or ABPV viruses, with exogenous segment inserted at Sites 1 and 2, or at Sites 8 and 6, or at any other combination of two or more Sites.

As used herein, the terms “Site 1,” “Site 2,” . . . “Site 8” are defined with reference to FIG. 3, which shows a schematic representation of the CrPV Group 1 IRES (which is representative of Group 1 IRESs in members of Dicistroviridae). “Site 1” refers to the region of the IRES that is 5′ to the first stem in Loop 1, whereas “Site 2” refers to the region between the second stem and the pseudoknot (PK1) in Loop 1. “Site 3” refers to the internal loop present between the first and second stems in Loop 1. “Site 4” refers to the region 5′ to the first stem in Loop 2, and “Site 5” refers to the region between the first hairpin and the immediately following stem in Loop 2. “Site 6” refers to the single-stranded region between the last stem of Loop 2 and PK1. “Site 7” similarly refers to the single-stranded region between PK1 and the first stem of Loop 3. Finally, “Site 8” refers to the single-stranded region 3′ to pseudoknot 3 (PK3).

In some aspects, the first and second sites respectively comprise: Site 1 and Site 2, Site 1 and Site 8, Site 2 and Site 7, Site 6 and Site 7, or Site 8 and Site 6. In some aspects, the first and second sites respectively comprise: Site 6 and Site 7, or Site 8 and Site 6. In some aspects, the first and second sites respectively comprise Site 6 and Site 7. In some aspects, the first and second sites respectively comprise Site 8 and Site 6.

In some aspects, the exogenous nucleotide sequence inserted at one or more of Sites 1-8 may comprise a first nucleotide sequence that is the reverse complement of at least a portion of the nucleotide sequence of a separate trigger RNA molecule. This first nucleotide sequence may be, e.g., 25-80 nt in length. Such constructs may further include a second exogenous nucleotide sequence inserted at a different site selected from Sites 1-8, which comprises a second nucleotide sequence which is the reverse complement of at least a portion of the first nucleotide sequence. This second nucleotide sequence may be, e.g., 8-25 nt in length. In some aspects, this architecture will cause the recombinant IRES to fold into an inactivated state due to interactions between the first and second exogenous nucleotide sequences (e.g., these sequence will at least partially hybridize under in vitro or in vivo conditions due to the second exogenous nucleotide sequence including a segment that is complementary to at least a portion of the first exogenous nucleotide sequence, resulting in attenuation or total loss of the IRES's ability to initiate translation of an operably-linked protein sequence encoded downstream of the IRES. In some aspects, these constructs may be activated by the presence of the aforementioned trigger RNA molecule, which comprises a nucleotide sequence that is the reverse compliment of the first nucleotide sequence. In some aspects, the trigger RNA molecule may comprise an artificial nucleotide sequence (e.g., to activate translation in an industrial setting), whereas in other aspects this trigger RNA may comprise an endogenous mRNA produced by a eukaryote, prokaryote, or virus (e.g., allowing the IRES to be used as a sensor to detect the presence of a given organism). It is understood that any of the aforementioned nucleotide sequences may consist solely of RNA. However, in some aspects these constructs (e.g., the exogenous nucleotide sequence(s) inserted at one or more of Sites 1-8 and/or the trigger RNA molecule) may include non-RNA or modified RNA bases at one or more positions.

In some aspects, the second exogenous nucleotide sequence is the reverse complement of at least a portion of the first exogenous nucleotide sequence. In some aspects, the first exogenous nucleotide sequence comprises a first nucleotide sequence that is the reverse complement of at least a portion of the nucleotide sequence of a separate trigger RNA molecule. The first nucleotide sequence can be, e.g., 25-80 nt in length, or 40-50 nt in length. The second nucleotide sequence can be, e.g., 8-25 nt or 6-15 nt in length. In some aspects, the first nucleotide sequence is 2.5× to 8× longer than the second nucleotide sequence. In some aspects, this architecture will cause the recombinant IRES to fold into an inactivated state due to interactions between the first and second exogenous nucleotide sequences (e.g., these sequence will at least partially hybridize under in vitro or in vivo conditions due to the second exogenous nucleotide sequence including a segment that is complementary to at least a portion of the first exogenous nucleotide sequence, resulting in attenuation or total loss of the IRES's ability to initiate translation of an operably-linked protein sequence encoded downstream of the IRES. In some aspects, these constructs may be activated by the presence of the aforementioned trigger RNA molecule, which comprises a nucleotide sequence that is the reverse compliment of the first nucleotide sequence. In some aspects, the trigger RNA molecule may comprise an artificial nucleotide sequence (e.g., to activate translation in an industrial setting), whereas in other aspects this trigger RNA may comprise an endogenous mRNA produced by a eukaryote, prokaryote, or virus (e.g., allowing the IRES to be used as a sensor to detect the presence of a given organism). It is understood that any of the aforementioned nucleotide sequences may consist solely of RNA. However, in some aspects these constructs (e.g., the inserted exogenous nucleotide sequence(s) and/or the trigger RNA molecule) may include non-RNA or modified RNA bases at one or more positions.

In some aspects, the second nucleotide sequence further comprises an IRES pseudoknot sequence. In some aspects, the second nucleotide sequence further comprises an IRES pseudoknot sequence, e.g. a naturally-occurring IRES pseudoknot sequence obtained from a wild-type IRES, including the wild-type IRES being modified as described herein. In some aspects, the second nucleotide sequence is inserted into an IRES pseudoknot sequence. IRES pseudoknot structures and sequences are known in the art.

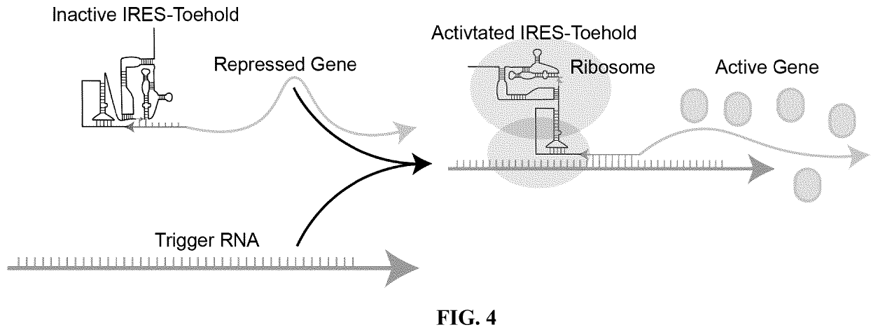

FIG. 4 illustrates the mechanism of operation underlying the recombinant IRES constructs described herein. In this example, an mRNA comprising a recombinant IRES according to the disclosure is shown to be operably-linked to a downstream segment encoding a protein of interest. Translation of the protein of interest is initially repressed because the recombinant IRES includes exogenous sequences at two different insertion sites (i.e., selected from Sites 1-8, defined above) which render the IRES inactive. As explained above, hybridization between the exogenous nucleotide sequences inserted at these sites disrupts the secondary structure of the recombinant IRES (i.e., maintaining the expression switch in the “OFF” state). However, as shown by this figure, when a trRNA is provided, the recombinant IRES switches “ON,” activating translation. The trRNA includes a segment that is a reverse complement of the nucleotide sequence inserted at the first of the two modified sites, and will consequently hybridize with that nucleotide sequence. In doing so, the trRNA disrupts the initial hybridization between the two exogenous nucleotide sequences, allowing the recombinant IRES to refold into an activated state.

In some aspects, recombinant IRES constructs according to the disclosure are incorporated into mRNA transcripts produced by a T7 RNA polymerase (e.g., such constructs may be downstream of and operably-linked to a T7 promoter sequence). The T7 polymerase may be produced by the plant cell (e.g., expressed from genomic DNA of the cell or from a plasmid) or introduced into the plant cell. In other aspects described herein, the recombinant IRES construct may be incorporated into an mRNA transcript produced by an alternative polymerase (e.g., a plant RNA polymerase II).

As noted above, the recombinant IRES constructs described herein may be incorporated into mRNA transcripts produced by a viral RNA polymerase (e.g., T7 polymerase, which does not apply a 5′ cap) because these constructs are able to recruit a ribosome and initiate translation. However, in some cases it may be undesirable to use a viral polymerase (e.g., a host cell may not produce T7, requiring co-transfection with a vector to supply this enzyme). Alternatively, it may be desirable to use endogenous RNA polymerase II for transcription in order to design a riboswitch system that uses a reduced number of exogenous components.

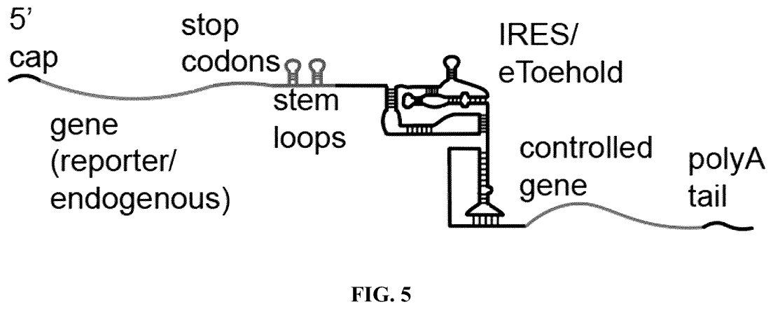

FIG. 5 is a schematic representation of an mRNA produced by RNA polymerase II which incorporates a recombinant IRES according to the disclosure. A illustrated by this schematic, the mRNA comprises a segment encoding a first protein, followed by a set of stop codons. A recombinant IRES according to the disclosure is present downstream from this element, and operably-linked to a segment encoding a second protein. Note that the mRNA transcript has a 5′ cap and a polyA tail, resulting from transcription by RNA polymerase II in this case. Translation of the second protein is controlled by the recombinant IRES, as is the case with constructs according to other aspects described herein. However, this configuration may be preferable in some instances due to its reliance on an endogenous eukaryotic mRNA promoter and polymerase, rather than viral components. Furthermore, as discussed in the examples below, this configuration appears to display reduced leakiness of expression compared to exemplary aspects which omit the upstream gene of interest.

Accordingly, in one aspect, described herein is a recombinant mRNA molecule, comprising: a first segment encoding a first protein; a second segment, downstream of the first segment, encoding a recombinant viral internal ribosome entry site (IRES) that has been modified to incorporate exogenous nucleotide sequences at a first site and a second site; and a third segment encoding a second protein, downstream from and operably linked to the second segment such that translation of the second protein is repressed when the IRES is in an inactivated state; wherein transcription of the recombinant mRNA molecule is dependent on a polymerase, and wherein the first site comprises a first nucleotide sequence, and the second site comprises a second nucleotide sequence which is the reverse complement of at least a portion of the first nucleotide sequence.

In various aspects, a nucleic acid sequence encoding a protein, located either 5′ or 3′ of a recombinant viral IRES riboswitch described herein can encode a protein which is a reporter protein, e.g., which produces a detectable signal. A reporter protein is a polypeptide with an easily assayed enzymatic activity or detectable signal that is naturally absent from the host cell. Exemplary but non-limiting reporter proteins include lacZ, catalase, xylE, GFP, RFP, YFP, ySUMO, CFP, EYFP, ECFP, mRFP1, mOrange, GFPmut3b, OFP, mBanana, neomycin phosphotransferase, luciferase, mCherry, and derivatives or variants thereof. In some aspects, the reporter protein is suitable for use in a colorimetric, luminescence, or fluorescence assay.

The recombinant IRES riboswitches described herein can be used as sensor modules, e.g., to detect particular trRNAs. The recombinant IRES riboswitches can be designed such that the trRNA is a sequence present in a target eukaryotic organism (e.g., a plant), a target prokaryotic organism, or a target virus. In the presence of the target organism/virus, or the target organism/virus in a particular transcriptional state, the recombinant IRES riboswitch will assume an active state and the protein encoded 3′ of the modified IRES sequence (e.g., a reporter protein) will be expressed, indicating the presence of the target. Such sensor systems are demonstrated herein, e.g., in Example 3 where infection with a number of different viruses is detected. In some aspects, the target prokaryotic organism or target virus is a pathogen, e.g., a plant pathogen such as Rhizoctonia, Colletotrichum, Phytophthora nicotianae. In some aspects, the target virus is Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus, Potato virus X, Citrus tristeza virus, Barley yellow dwarf virus, Potato leafroll virus or Tomato bushy stunt virus. In some aspects, the target prokaryotic or eukaryotic organism can be an organism can be an organism comprising and/or expressing the recombinant IRES riboswitches and the trRNA can be a non-constitutively expressed RNA, e.g., an RNA expressed only at certain developmental or differentiation stages, or a RNA expressed in response to certain stimuli and/or stresses.

### Recombinant Cells Engineered to Incorporate Riboswitch Modules

In some aspects, the disclosure provides plant cells engineered to express proteins under the control of the recombinant IRESs described herein.

In some aspects, the plant cell may comprise genomic DNA encoding a recombinant IRES. In others, the recombinant IRES may be encoded by a vector (e.g., a plasmid) present within the plant cell. The recombinant IRES may be operably-linked to an endogenous or exogenous promoter and/or a gene encoding a protein of interest.

### Use of Riboswitch Modules in Cell-Free Expression Systems

In some aspects, the recombinant IRES modules described herein may be used in cell-free expression systems. For example, a kit or assay may utilize a cell-free lysate produced from plant cells that includes DNA encoding at least one mRNA which incorporates a recombinant IRES module. In other aspects, such kits or assays may include transcribed mRNAs that incorporate at least one recombinant IRES module. It is understood that that the riboswitch mechanism described herein may be used as a sensor to trigger expression of a protein of interest in a variety of in vitro applications (e.g., as a sensor to detect the presence of viral mRNA).

### Modulating Translation in Eukaryotes or Cell-Free Expression Systems Using Recombinant IRES Riboswitch Modules

The recombinant IRES riboswitch modules described herein may be used to modulate the expression of a protein of interest, e.g., in a plant cell or in a cell-free expression system.

In some aspects, a plant cell may be transfected with a vector that encodes a protein of interest operably-linked to an upstream RES riboswitch according to the disclosure. The IRES riboswitch may comprise a sequence sharing at least 90, 95, 98, 99 or 100% sequence identity with that of a viral IRES, except for the presence of exogenous nucleotide sequences at two sites. In some aspects, the IRES riboswitch may comprise a sequence sharing at least 90, 95, 98, 99 or 100% sequence identity with that of a Group I Dicistroviridae IRES (e.g., the CrPV IRES represented by SEQ ID NO: 1), except for the presence of exogenous nucleotide sequences at two sites (e.g., any combination of Sites 1-8, as defined above). This pair of exogenous sequences may comprise a first nucleotide sequence that is 25-80 nt in length and a second nucleotide sequence that is 8-25 nt in length, wherein second nucleotide sequence is the reverse complement of a portion of the first nucleotide sequence, causing the pair of exogenous sequences to hybridize. As a result of this hybridization, the IRES riboswitch assumes an inactive fold, preventing translation of the downstream protein of interest. Translation may be activated by introducing a trRNA which comprises a nucleotide sequence that is the reverse complement of the first nucleotide sequence, causing the first nucleotide sequence to hybridize with the trRNA rather than the second nucleotide sequence, and consequently allowing the IRES riboswitch to assume an active fold. In some aspects, the trRNA may be introduced by transfection or expressed by a vector.

In some aspects, the trRNA may be configured to have a unique sequence that is not found in mRNAs expressed by the plant cell used for expression. The selection of a unique sequence may reduce or eliminate off-target effects (e.g., unintended hybridization between the trRNA and other endogenous mRNAs produced by the plant cell).

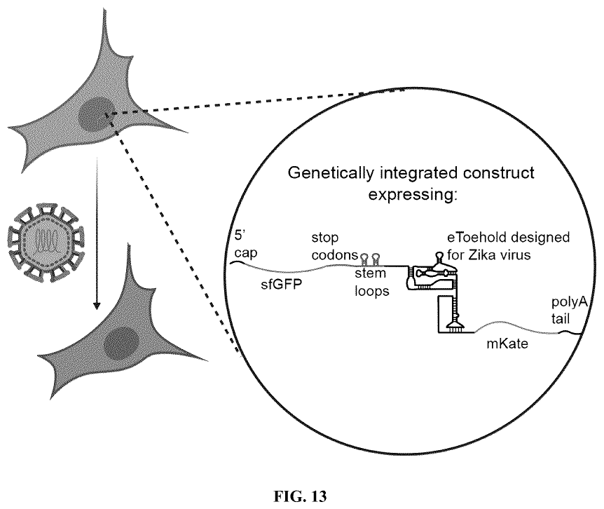

In some aspects, the trRNA may comprise a portion of an mRNA expressed by the plant cell or an external stimulus (e.g., a viral mRNA produced following infection of the cell by a virus, as shown by FIG. 13). In some aspects, the concentration of the trRNA may be increased or decreased to modulate expression of the protein of interest.

The mRNA comprising the IRES riboswitch may be operably-linked to a promoter suitable for expression in the selected plant cell. In some aspects, a T7 promoter may be used (e.g., if the selected plant cell is engineered to produce T7 polymerase). Alternatively, a eukaryotic promoter (e.g., an RNA Polymerase II promoter) may be used. The selection of a suitable promoter will vary depending on the intended application of the IRES riboswitches described herein. For example, an inducible promoter may be desirable in some applications, whereas a constitutive promoter may be desired in others. Some promoters may also allow for tighter control over expression of the mRNA (e.g., a T7 promoter may be leaky when used in a eukaryotic cell due to low-level recruitment of RNA polymerase II).

In some aspects, the IRES riboswitch can be operably-linked to one or more of: a) an IRES pseudoknot sequence; b) an IRES pseudoknot sequence found in the wild-type sequence of a virus in which the IRES naturally occurs; c) a promoter and/or upstream activating factor binding sequence; d) a stop codon; e) a stem-loop (e.g., SEQ ID NO: 9); f) a 5′ cap; g) a reporter gene; and h) a poly-A tail, wherein the one or more of elements a-h are individually located 5′ or 3′ of the IRES riboswitch. In some aspects, the IRES riboswitch can be operably-linked to one or more of: a) an IRES pseudoknot sequence; b) an IRES pseudoknot sequence found in the wild-type sequence of a virus in which the IRES naturally occurs; c) a promoter and/or upstream activating factor binding sequence; d) a stop codon; e) a stem-loop; f) a 5′ cap; and g) a reporter gene, wherein the one or more of elements a-g are individually located 5′ of the IRES riboswitch.

A promoter for use in the methods and compositions described herein can be an RNA polymerase II; a polymerase other than RNA polymerase II; a T7 polymerase; a T3 promoter, a araBAD promoter, a trp promoter, a lac promoter, a Ptac promoter, a pL promoter, and/or an SP6 polymerase. An upstream activating factor binding sequence can be the upstream activation factor binding DNA sequence (UAF2) from Saccharomyces cerevisiae (e.g., SEQ ID NO: 10).

In one aspect, described herein is a vector, e.g., a plasmid or viral vector comprising the recombinant nucleic acid molecule, expression construct, recombinant mRNA molecule, or recombinant IRES riboswitch described herein. In one aspect, described herein is a eukaryotic cell (e.g., a plant cell) comprising DNA encoding a recombinant nucleic acid molecule, expression construct, or recombinant mRNA molecule described herein, wherein the DNA is: integrated into the genomic DNA of the eukaryotic cell, or present on a vector (e.g, a plasmid or viral vector) present within the eukaryotic cell. Such cells are considered to be engineered by the introduction of the recombinant nucleic acid molecule, expression construct, recombinant mRNA molecule, or recombinant IRES riboswitch.

It is understood that the IRES riboswitches described herein may also be used in cell-free expressions. For example, a kit may include mRNA comprising an IRES riboswitch operably linked to a segment encoding a protein of interest, as well as components needed for in vitro protein expression (e.g., a cellular lysate).

The IRES riboswitches described herein may be used in a variety of industrial and agricultural applications. IRES riboswitches may also be used as a means to control cell differentiation. For example, a plant stem cell may be engineered to incorporate an IRES riboswitch triggered by an mRNA produced by a specific cell type, wherein the riboswitch controls expression of a toxin or a protein that induces apoptosis. Such mechanisms may be used to maintain the purity of a stem cell line by eliminating undesirable cell types which may be produced inadvertently.

In the agricultural context, IRES riboswitches may be used, e.g., to develop plants that respond to specific bacterial, fungal, viral, or insect pests, or to environmental conditions. For example, a plant may be engineered to incorporate an IRES riboswitch which controls the expression of a pesticidal protein, with the trRNA being a portion of an mRNA expressed by a bacterial or fungal parasite. Upon infection, the plant's cells would begin (or increase) expression of the pesticidal protein. Similar configurations may be used to engineer plants that express proteins capable of mitigating stress or increased growth in response to environmental triggers. For example, a plant may be engineered to increase production of a protein that modulates growth when triggered by an mRNA that is upregulated in response to specific environmental conditions (e.g., an mRNA encoding a heat shock protein).

In some aspects, the IRES riboswitches described herein can be used in a method of detecting viral infection of a cell. For example, a eukaryotic cell (e.g., a plant cell) may be engineered to express a recombinant nucleic acid comprising an IRES riboswitch as described herein, wherein the first nucleotide sequence of the recombinant nucleic acid molecule is configured to be the reverse compliment of at least a portion of a mRNA sequence unique to a virus; and it is determined whether the eukaryotic cell is infected with the virus by detecting and/or measuring the presence of the protein encoded by the second segment of the recombinant nucleic acid molecule, wherein the eukaryotic cell is not a plant cell. The virus may be, e.g., any of the plant viruses described herein.

In other aspects, the IRES riboswitches described herein can be used in a method of controlling or monitoring differentiation of a eukaryotic cell (e.g., a plant cell). For example, a eukaryotic cell may be engineered to express a recombinant nucleic acid comprising an IRES riboswitch as described herein, and cultured, wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of an undesired cell type.

In some aspects, described herein is a kit or system, comprising one or more of: a plasmid or viral vector, a recombinant nucleic acid molecule, expression construct, recombinant mRNA molecule, recombinant IRES riboswitch, and/or trRNA as described herein. A kit is an assemblage of materials or components, including at least one of the foregoing elements described herein. The exact nature of the components configured in the kit depends on its intended purpose. In some aspects, a kit includes instructions for use. “Instructions for use” typically include a tangible expression describing the technique to be employed in using the components of the kit, e.g., to detect an organism or RNA. Still in accordance with the present invention, “instructions for use” may include a tangible expression describing the preparation of a recombinant IRES riboswitch, cell, or expression system described herein such as reconstitution, dilution, mixing, or incubation instructions, and the like, typically for an intended purpose. Optionally, the kit may also contain other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition described herein. The packaging material generally has an external label which indicates the contents and/or purpose of the kit and/or its components.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some aspects, the expression of nucleic acid sequence and/or protein described herein is/are tissue-specific. In some aspects, the expression of a nucleic acid sequence and/or protein described herein is/are global. In some aspects, the expression of a nucleic acid sequence and/or protein described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some aspects, the methods described herein relate to measuring, detecting, or determining the level of at least one target. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some aspects, measuring can be a quantitative observation.

In some aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

The term “exogenous” refers to a substance present in a cell or nucleic acid sequence other than its native source. The term “exogenous” when used herein can refer to a nucleic acid (e.g. a nucleic acid encoding a polypeptide) or a polypeptide that has been introduced by a process involving the hand of man into a nucleic acid molecule or biological system such as a cell or organism in which it is not normally found and one wishes to introduce the nucleic acid or polypeptide into such a nucleic acid molecule, cell or organism. In contrast, the term “endogenous” refers to a substance that is native to the nucleic acid molecule or biological system or cell.

In some aspects, a nucleic acid as described herein is comprised by a vector. In some of the aspects described herein, a nucleic acid sequence as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some aspects, the vector or nucleic acid is recombinant, e.g., it comprises sequences originating from at least two different sources. In some aspects, the vector or nucleic acid comprises sequences originating from at least two different species. In some aspects, the vector nucleic acid comprises sequences originating from at least two different genes. A sequence can be modified to be recombinant, or a sequence can be integrated into another sequence to provide a recombinant sequence by methods well known in the art, e.g., through use of restriction enzymes and ligases.

In some aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some aspects, the expression system is an organism other than the source of the native/wild-type sequence (or a cell obtained from such organism). In some aspects, the vector and/or nucleic acid sequence described herein is codon-optimized for expression in a plant (e.g., in a tobacco or soybean plant) or in a particular plant cell or tissue (e.g., in the roots or leaves of a plant). In some aspects, the vector and/or nucleic acid sequence described herein is codon-optimized for expression in a plant cell. In some aspects, the vector and/or nucleic acid sequence described herein is codon-optimized for expression in a yeast or yeast cell. In some aspects, the vector and/or nucleic acid sequence described herein is codon-optimized for expression in a bacterial cell. In some aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in plant cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some aspects, be combined with other suitable compositions and therapies. In some aspects, the vector is episomal. The use of a suitable episomal vector provides a means of maintaining the nucleotide of interest in the subject in high copy number extra chromosomal DNA thereby eliminating potential effects of chromosomal integration.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some aspects, contacting comprises physical activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±10%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20^{th} Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

### EXAMPLES

The following non-limiting examples are provided to further illustrate the present disclosure.

### Example 1: Screening for Recombinant IRES Riboswitches

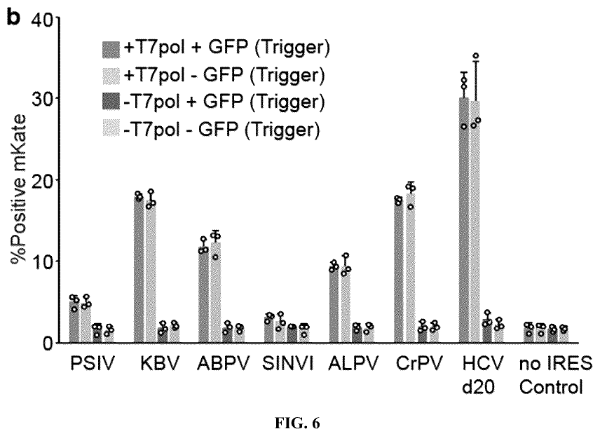

The process of designing recombinant IRES riboswitches began with the selection of IRES modules from viral databases and testing them in a human embryonic kidney 293 (HEK293) cell-based transfection assay. Co-transfected with these constructs, the T7 polymerase was found not to 5′ cap mRNA, resulting in a dramatic enhancement of mKate expression by the IRES modules, as illustrated by FIG. 6. The IRES of Hepatitis C virus (HCVd20) was used as a control for this assay, but was not pursued due to its extensive structural differences compared to the rest of the selected IRES modules and reported reliance of activity on small RNAs. Based on this initial screen, the cricket paralysis virus (CrPV), kashmir bee virus (KBV), and acute bee paralysis virus (ABPV) IRES modules were selected for further study, with focus on the CrPV IRES due to the abundance of existing structural information.

In order to test whether disruption of the structure of these IRES modules could reduce their translational initiation capability, variants of the CrPV, KBV and ABVP IRES modules, which incorporated exogenous nucleotide sequences, were engineered. It was theorized that the insertion of two reverse complement segments of DNA into two sites of the IRES module could be used to distort the functional configuration/structure of the IRES module (e.g., due to hybridization of these two segments). The two reverse complement segments of DNA were further designed to be released by a trigger RNA sequence (trRNA) that is the reverse complement of one of the segments and which includes a longer overlapping portion compared to the overlap between the first and second segments. Specifically, the longer segment of inserted DNA (40-50 base pairs) was the reverse complement of a portion of the target trigger and the shorter segment (10-15 base pairs) was the reverse complement of a portion of the first segment. Eight sites were selected where insertions would not individually break IRES module activity (i.e. Sites 1-8 shown in FIG. 3) and avoided Loop 3, whose complete functionality is crucial for any level of IRES module activity.

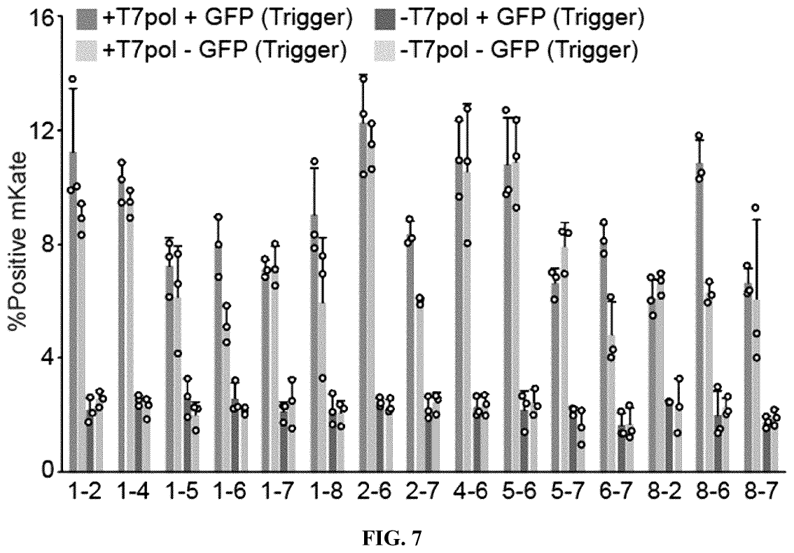

A set of 15 fold combinations were tested using the same assay for IRES activity determination (FIG. 7). Each fold combination was named using the format (long segment site number−short segment site number). In this initial study, GFP mRNA was used as a trigger RNA. Although folds 1-2, 1-8, and 2-7 led to reproducible fold changes in this initial screen, further testing showed a lack of transferability of the same folds towards other target triggers. It was decided to continue engineering folds 6-7 and 8-6, which both showed 1.7× fold increases in percentage of mKate positive cells when the trigger RNA was transfected concurrently.

### Example 2: Optimization of Recombinant IRES Riboswitches

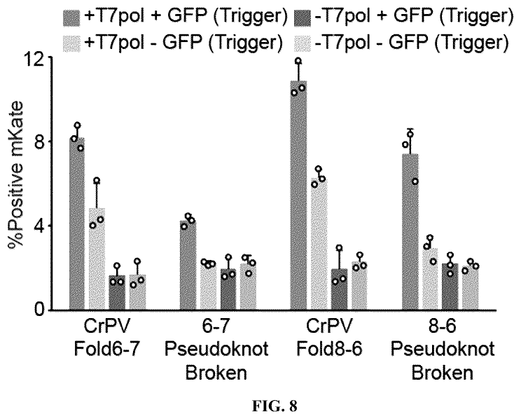

Practical applications of the presently disclosed recombinant IRES riboswitches will often require high fold-changes of downstream protein translation following trigger induction. Therefore, a reduction in the leakiness of the two hits from the initial screen (i.e., folds 6-7 and 8-6) was sought. Previous studies suggest that IRES pseudoknots are critical for ribosome recruitment. As such, a study was conducted to determine whether the distortion of an IRES module along with the breaking of the pseudoknot at the same locations (after insertion site 7 for fold 6-7 and after insertion site 6 for fold 8-6) would reduce the leakiness of the IRES module. New recombinant IRES riboswitches were designed such that the base pairs in insertion 1 following where insertion 2 would anneal were reverse complements of the base pairs corresponding to the pseudoknots. In the absence of a trigger, this annealing would prevent correct pseudoknot folding, i.e., creating a pseudoknot breaking site (PB site). Designing recombinant IRES riboswitches with the PB site led to a reduction of the “OFF” (no trigger) state to levels observed when no IRES module was present and increased the “ON” (with trigger) to “OFF” (without trigger) fold-change from 1.7× to 2.5× for fold 8-6, as shown by FIG. 8. Further optimization of the melting temperature of the annealing portion showed a dependence of IRES riboswitch behavior on this parameter but no improvements in the “ON” to “OFF” fold-change.

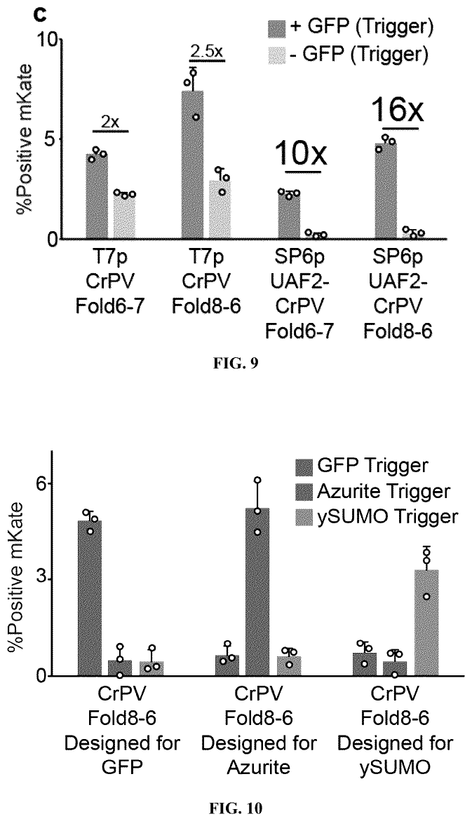

Based on these findings, it was theorized that the remaining leakiness was caused by non-specific binding of the T7 promoter sequence by endogenous RNA polymerase II, leading to 5′ capping and bypassing of IRES-mediated translational initiation. To find a suitable polymerase-promoter replacement pair, we placed sfGFP under different promoters with high RNA expression levels and transfected HEK293T cells (without corresponding polymerases). It was found that P_{SP6} was significantly less leaky than P_{T7} and its analogues. Upstream activation sequences were tested for RNA polymerase I, which have been shown to decrease RNA polymerase II binding. Our findings demonstrated that an upstream activation factor binding DNA sequence from Saccharomyces cerevisiae decreased leakiness (named UAF2). By combining these two methods/techniques/approaches, we minimized leakiness substantially and reached an “ON” to “OFF” trigger-mRNA based induction of 15.9× fold, as shown by FIG. 9. Similar folds of induction could be reached by adding stop codons and stem loops before the recombinant IRES riboswitch. Further increases in “ON” to “OFF” fold change could be achieved using a recombinant IRES riboswitch that hides a kozak sequence downstream of the riboswitch, but this method requires extensive screening (only 1 out of 9 kozak toeholds improved fold change).

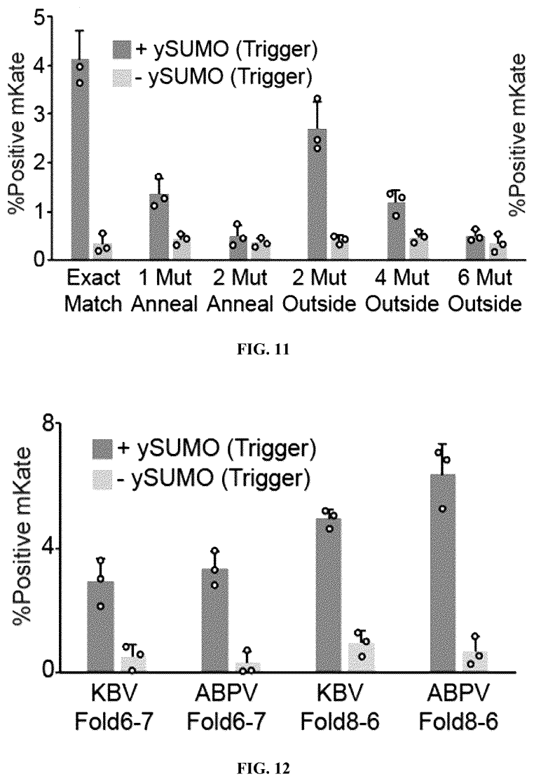

To further characterize the recombinant IRES riboswitch of the present disclosure, we tested the capability of these riboswitches to sense trigger mRNA from a number of sources. In particular, we designed folds to detect GFP, Azurite, and yeast SUMO mRNA. By exposing each of the folds to each of the three types of mRNA, we demonstrate that the recombinant IRES riboswitch are orthogonal and can be designed for most target mRNAs (FIG. 10). We further tested the sensitivity of our folds by mutating the insertions both inside insertion 1 and 2 (Mut Anneal) or in insertion 1 but not insertion 2 (Mut Outside). We found that recombinant IRES riboswitch are very sensitive to mutations within insertion 2, indicated by 3× fold decreases in activation level with a single mutation, but not as sensitive to mutations outside of insertion 2 (FIG. 11). This study therefor confirmed that recombinant IRES riboswitches can be designed for different mRNAs while maintaining specificity.

We then aimed to expand possible PB sites for easier recombinant IRES riboswitch design. Given the sequence differences and structural similarities between the IRES modules, we sought to achieve this by creating folds using different IRES modules. In creating recombinant IRES riboswitches using both KBV and ABPV IRES modules, we observed similar fold changes of trigger-induced translation to the CrPV IRES modules (FIG. 12). These alternative IRES eToeholds effectively expand the range of possible PB sites.

### Example 3: Use of Recombinant IRES Riboswitches in Agricultural Applications

The recombinant IRES riboswitches described herein may be used in different plant systems, including Arabidopsis, tobacco, and soybean. It is further believed that these recombinant IRES riboswitches (and the related methods) discussed herein may be applied to maize, canola, soybean, cotton, wheat, rice, and other agriculturally-significant crops. The demonstration of functionality in plants is a novel application of the recombinant IRES riboswitches described herein, as there is no comparable system available in art offering flexible translational regulation in plants. The use of recombinant IRES riboswitch switches in plants offers completely new ways of protein regulation thereby enabling multiple new ways of regulating trait expression.

The recombinant IRES riboswitches described herein may be used in plants based in accordance with the following exemplary protocol. First, a recombinant IRES riboswitch construct can be designed based upon a CrPV, KBV, ABPV or PSIV IRES module, with a desired pair of exogenous nucleotide sequences inserts for use with a selected trRNA. This construct may be driven, e.g., by a Cauliflower mosaic virus (CaMV) 35S promoter with luciferase used as the reporter. To further improve the functionality of this novel regulation system, one can decrease the potential leakiness that might be caused by the endogenous RNA polymerase II in plants by incorporation of two stop codons and two stem loops in the construct. The construct can be introduced into a plant (e.g., introduced into tobacco leaves or transformed into Arabidopsis) by standard methods. The luciferase activity can be monitored in the leaves or other plant tissue by measuring luminescence. Consistent with what we observed in HEK293T cells, we expect recombinant IRES riboswitches based on the CrPV or KBV IRES modules to result in higher expression of luciferase than the negative control construct (without the IRES sequence). This would demonstrate the functionality of recombinant IRES riboswitches in plants (positive control).

One may also design recombinant IRES riboswitches with PB sites, as described above, that use three soybean root specific genes as triggers. These riboswitches can be also driven by CaMV 35S promoter and may contain two stop codons and two stem loops as in the controls. The switches can be tested, e.g., in tobacco leaves through infiltration, and in Arabidopsis and soybean through agrobacterium transformation. Since there is no homolog for these genes in Arabidopsis or tobacco, it is expected that the riboswitches would turn off luciferase expression in tobacco and Arabidopsis, and the soybean non-root tissues where there is extremely low expression of these genes. We expect to only observe the expression of luciferase at the basal level, similar to the expression level of the negative control construct without the IRES sequence. This would demonstrate the tightness of the regulatory system and establishes the “OFF” state of the tested riboswitches.

In other experiments, one may infiltrate tobacco and transformed Arabidopsis with the constructs that contain IRES riboswitches as well as the corresponding root-specific genes to turn on these switches. With the presence of the trigger, one would expect to observe a 2-5× fold increase in luciferase expression compared to the constructs without the trigger in tobacco and Arabidopsis. We would also expect to observe an expression level of luciferase in root tissue that is 3-7× fold higher than that of non-root tissues, due to the presence of the trigger mRNA in the root tissue. Still, the “ON” to “OFF” fold change may not be as high as what was observed in HEK293T cells. This could be due to the temperature effect, as the culture temperature of HEK293T was 37° C., whereas the temperature for the plant growth chamber was 25° C. If so, one may then optimize the riboswitches by lowering the annealing temperature between the 40-50 nt strand used at the first insertion site and the 10-15 nt strand used in the second insertion site (see FIG. 4). After optimization, the luciferase expression level in the presence of the trigger is expected to be 8-12× fold higher than that observed in the absence of the trigger. These experiments may be carried out to demonstrate that the recombinant IRES riboswitches described herein can be used in plants to turn on and off gene expression based on the presence and absence of the trigger, respectively.

Currently, all traits are expressed constitutively in a whole plant. On-demand and local expression of select traits could save energy for the plants, and thus may improve the yield of the transgenic crops. Respective tissue specific expression utilizing tissue or organ specific promoters like root, root hair, parenchyma, leaf, guard cell, meristem, flower, anther, seed coat, seed embryo specific promoters are known to the person skilled in the art. The recombinant IRES riboswitches of the present disclosure may be used as flexible tool to control trait expression in plants at the translational level. One or more of these present riboswitches can be integrated into a plant to regulate the expression of multiple traits, if desired, allowing for more efficient engineering of existing and novel pathways.

### Example 4: Induction of Recombinant IRES Riboswitch-Regulated Genes for Trait Expression in Plants

A recombinant IRES riboswitch may be used to modulate a variety of plant traits. For example, these riboswitches can be used to tightly regulate disease defense genes. One may design a recombinant IRES riboswitch that recognizes fungal or bacterial specific RNA molecules, which are transferred upon infection of the plant cell. During normal plant growth using a constitutive promoter (e.g., CaMV 35S or USP), protein expression of defense genes (e.g., defensins, chitinases, cell death genes) would be blocked by the recombinant IRES riboswitch. This would reduce energy expenditure and resource consumption for the plant as amino acids for protein expression are significant nitrogen sources. However, upon infection by fungi or bacteria, the trigger RNA will be introduced and will interact with the recombinant IRES riboswitch(es) and directly trigger protein expression of the respective defense mechanism(s), allowing immediate activity compared to the delayed response of triggering translation and then transcription. Therefore, the recombinant IRES riboswitches described herein allow precise mediation of a disease attack. Using Asian Soybean Rust (ASR) as an example, one can identify the RNA molecules that ASR injects into soybean during infection, and design a recombinant IRES riboswitch and trigger RNA based on the sequences of these RNAs. The riboswitch can then be inserted upstream of the Resistance genes (R-genes) to block the expression of the R-genes in the absence of ASR. During ASR infection, the riboswitches will be activated by the ASR RNAs to trigger the expression of R-genes, thus conferring resistance to ASR. As discussed above, stop codons and stem loops can be inserted to reduce leakiness, and the annealing temperature can be optimized, to decrease the background expression of the R-genes and to allow the maximal induction.

In another embodiment, the recombinant IRES riboswitch can be used to tightly regulate expression of pest control traits only upon infestation of insects. Insect infestation can lead to upregulation of specific genes. For example, Hevein-like protein (HEL) is induced by Pieris rapae's feeding on Arabidopsis, but not induced by wounding. Similar genes can be identified in crops in response to insect feeding. Recombinant IRES riboswitches can be designed to sense mRNA produced when these genes are transcribed so that the traits are only expressed upon infestation. Similar to the disease control application, this will save the plants energy and resource used for unnecessarily expressing the traits when there is no pest present. Therefore, the recombinant IRES riboswitches may help improve the yield of transgenic crops.

In another example, recombinant IRES riboswitches can be used to tightly regulate pest defense genes. Pest defense genes could be toxins specific to certain insects (e.g., Bt toxin). In this case, expression of the toxin is transcribed in the cell, but blocked from protein expression by the recombinant IRES riboswitch. Upon digestion of plant material by a damaging pest e.g. a Lepidopteran caterpillar, the mRNA is taken up by insect gut cells. Upon interaction with specific RNA molecules present in the gut cells, the recombinant IRES riboswitch may be configured to activate translation of the toxin mRNA, allowing specific and precise action. By using the recombinant IRES riboswitch, a species-specific release mechanism can be constructed to allow activity only against targeted pest, but not against beneficial insects such as bees. Another advantage of this application is that the toxins are not expressed as proteins in the crops, and thus will be more favorable to consumers concerned by transgenic crops.

In another example, a recombinant IRES riboswitch can be used to tightly regulate trait expression in specific tissues of crops or at different development stages of crops. Some pests only infest crops at certain development stages, or only feed on certain tissues of crops (e.g., caterpillars only feeding on leaves or corn ears, stink bugs only feeding on fruits, and nematodes only feed on roots). Designing recombinant IRES riboswitches to be turned on by mRNA associated with development stage-specific genes or tissue-specific genes can tightly regulate the trait expression only at the specific development stage or at specific tissue, respectively. This again may improve yield of transgenic crops through energy and resource saving. It may also provide a way to eliminate the presence of the traits in the final products going to the market, and thus will have better societal acceptance of the transgenic crops. Tissue-specific expression of traits can also avoid expression of traits in flowers to protect the beneficial insects such as bees.

In another example, recombinant IRES riboswitches can be used to tightly regulate stress responses of plants in order to improve crop efficiency and yield. Abiotic stress (e.g., drought, nutrient deficiency) triggers multiple responses in plants, some of them reducing crop yield (e.g., the number of seeds produced under stressed conditions).

One example is the induction of the yield gene Os_PCD (SEQ ID NO: 2) by the endogenously expressed OsHsp70 gene (SEQ ID NO: 3). First, the recombinant IRES riboswitch is designed using the sequence of OsHsp70 (SEQ ID NO: 3). The 4 variable regions are selected based on distinctive sequence pattern analysis (e.g., using SeqAlign, BLAST, or other pattern analysis software known in the art) in order to avoid interaction with other heat-shock proteins or any similar mRNA transcripts in rice (Orzya sativa). Expression of OsHsp70 has been shown to be induced by heat stress (above 30 degree Celsius). Next, the Hsp70-riboswitch sequence may be cloned upstream of the start codon of Os_PCD gene (SEQ ID NO: 2). This cassette may then cloned downstream of the constitutive rice promoter APX (SEQ ID NO: 4) into a plant transformation vector (T-plasmid). Os_PCD is a Pterin-4a-carbinolamine dehydratases acting as bifunctional protein, which is both a transcription activator and a metabolic enzyme, acting as RuBisCO assembly factor. In addition to the Os_PCD gene cassette, the RNA polymerase II sequence may be included as described above under the control of a weak constitutive promoter (e.g., the CaMV 35S promoter, which shows weak expression in monocotylous plants). The construct is transformed into Orzya sativa cv. Indica by standard methods. The activity in plants can be described as following: Specifically, heat stress results in less stable RuBisCO enzyme, thereby affecting photosynthesis, thereby reducing the capacity of the plant for photosynthesis, thereby decreasing yield. The constitutive expression of Os_PCD results in the waste of energy and nutrients (e.g., nitrogen) for the plant. The use of the recombinant IRES riboswitche results in the blocked protein production of Os_PCD (SEQ ID NO: 5) under non-heat conditions. However, upon heat stress, OsHsp70 mRNA is expressed, triggering the recombinant IRES riboswitch to allow translation of the Os_PCD mRNA. The resulting Os_PCD protein can rapidly stabilize RuBisCO activity, thereby preventing yield loss. Once heat stress is reduced (e.g. temperature below 30 degree Celsius), OsHsp70 mRNA expression is reduced/stopped, causing the translation of Os_PCD to cease when the recombinant IRES riboswitch folds into an inactive state, saving resources of the plant. This generates a flexible, versatile and reversible system for stress responses.

In another embodiment, recombinant IRES riboswitches can be used to increase crop yield by generation of hybrids. One important technical necessity for successful hybrid generation is reversible male sterility. Recombinant IRES riboswitch allow for precise switching on or off of male sterility. For example, a plant can be engineered to express a recombinant IRES riboswitch in the anther and can be used to trigger expression of a non-specific RNAse, degrading RNA during anthesis, thereby blocking pollen formation. In this state, a normal and efficient propagation of the hybrid female is possible. Using either a chemical trigger (e.g., gibberellic acid or nitrogen) or abiotic changes (e.g., day length), the expression of cell-specific RNA is changed, triggering the degradation of pollen, thereby generating a male sterile flower.

One potential application of this technique is the regulation of flower formation in soybean to improve yield under stress conditions. Crop yield is defined by weight of seeds per area of planted crops. One important factor for yield in this calculation is the number of flowers to produce the seeds. In theory, the more flowers per plant, the more seeds per planted area. Under practical conditions there are several limitations to this theoretical calculation, namely the abortion or unfilling of pods in soybean under stress conditions. This limits yield despite flower formation. One potential solution is the induction of more flowers as done by breeding or using genome editing. However, the increase of flowers at the time of heat or drought stress might only lead to more aborted or unfilled pods, requiring a new solution. Recombinant IRES riboswitches offer the ability to time flowering induction to low stress impact windows and limit flowering during high stress impact, thereby increasing the success rate of flowers forming seeds.

For the precise induction of flowers in soybean, a plant transformation plasmid construct is designed similarly as described in the preceding examples. The variable regions of the recombinant IRES riboswitch may be designed to be triggered by Gm_HSF (SEQ ID NO: 6), a transcription factor down-regulated under heat and drought conditions, and may be cloned upstream of tomato SP5G (SEQ ID NO:7), a gene responsible for rapid flowering. The cassette is then cloned downstream of the constitutive PcUbi promoter for expression. The additional cassette for RNA polymerase II may be added to the transformation cassette as described above and the resulting T-plasmid ay be used for transformation in Glycine max. Upon testing under normal conditions, SP5G is inducing formation of flowers in soybean, increasing the number of seeds. Under heat or drought conditions, Gm_HSF transcript levels decrease, thereby causing the recombinant IRES riboswitch to inactivate, blocking the translation of SP5G (SEQ ID NO:8), and reducing flower formation until stress conditions decline and expression of Gm_HSF normalizes, activating the riboswitch once again and thereby translation of SP5G.

### Example 5: Evaluation of Exemplary Viral IRES Elements in Tobacco Plants

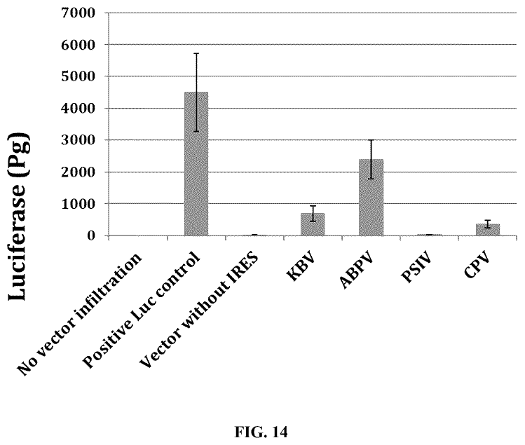

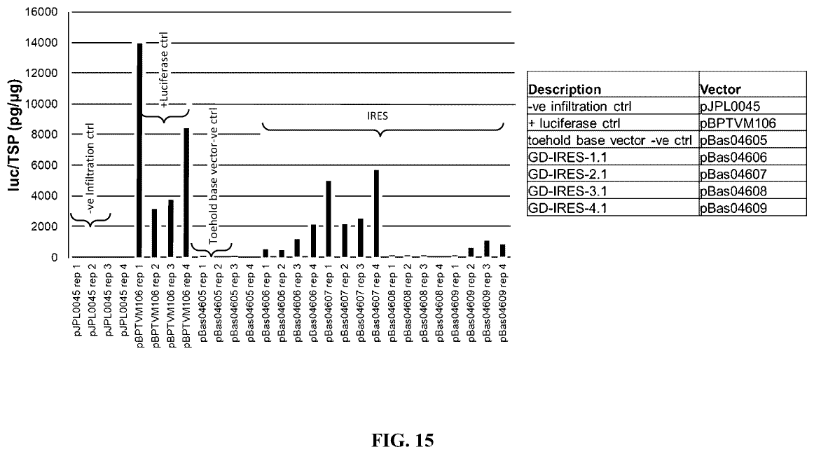

The IRES riboswitches described herein may be used as a platform to modulate gene expression in various eukaryotic organisms, including plants, as discussed in further detail above. Several experiments were conducted to assess the expression levels of exemplary viral IRES elements in tobacco plants, using agrobacterium-mediated transfection. Agroinfiltration is a common technique used in plant biology to induce transient expression of genes in a plant, in order to produce a desired protein or to express a recombinant construct. In short, a suspension of Agrobacterium tumefaciens is introduced into a plant leaf by direct injection or by vacuum infiltration. After infiltration, the bacteria transfer the desired gene into the plant cells via transfer of T-DNA. In this case, A. tumefaciens was used to generate modified tobacco plants. Each plant was transfected with plasmids configured to provide transient expression of mRNAs encoding an exemplary viral IRES element coupled to a downstream reporter (luciferase). Various controls were also tested. The relative effectiveness of these IRES elements was then measured using a luciferase assay. As evidenced by the data shown in FIGS. 14-15, exemplary viral IRES elements according to the disclosure are functional in tobacco. Similar results are expected in various other agriculturally-significant crops.

An initial set of expression vectors was designed to assess the effectiveness of different Dicistroviridae viral IRES elements. Each vector included a luciferase expression cassette under the control of the IRES from either KBV, ABPV, CrPV, or PsIV, as well as a GFP expression cassette for use as a control to monitor the transfection rate (the “Viral IRES Vector”).

Each Viral IRES Vector was transfected in a tobacco plant using agroinfiltration. In brief, an agrobacterium cell suspension was prepared by streaking agrobacterium cells transformed with the vector of interest on selective plates and incubated at 28° C. for 1-2 days until heavy cell growth appeared on the plates. The freshly grown agrobacterium cells were then re-suspended in co-cultivation media (CCM from PGE) with acetosyringone at an OD₆₀₀ of 0.3-0.5. The cells were incubated for at least 30 minutes at room temperature before agroinfiltration. Nicotiana benthamiana plants were prepared by growing N. benthamiana seedlings from seeds in a growth chamber (28° C. for 15 hours with light: 26° C. for 9 hours in the dark) for 1 week. The seedlings were then transferred to an individual pot and continue plant growth for another 2-3 weeks until 5-8 fully expanded leaves appear. For each vector, agroinfiltration was performed by infiltrating 2 fully-expanded leaves of 2 tobacco plants with the agrobacterium cell suspension, by pushing the agrobacterium suspension into the airspaces inside the leaf from the underside using a 1 mL syringe without a needle. The region of infiltration was marked and the plants were then placed back in the growth chamber for 2 days. Sample leaf punches were later collected to analyze the transient expression of the vector constructs.

As noted above, each viral IRES was used to control expression of a luciferase cassette. Thus, the effectiveness of each viral IRES was assessed using a luciferase assay (specifically, the “Steady-Glo® Luciferase Assay System” (Promega® catalog #PR-E2520). In brief, leaf punches were collected after 3 days and temporarily stored at −80° C. in sealed 96-well culture plates (two punches per well). When it was time to perform this assay, the culture plates were removed from the freezer and placed on dry ice. For each test sample, two glass beads were added to the well, along with 150 μl of 1×PBS with protease inhibitor. The sample was pulverized for one minute using a bead beater. The plate was then centrifuged at 4,000 RPM for 10 minutes at 4° C. to spin-down the pulverized sample. Afterward, 25 μl of supernatant was aliquoted to the well of a 96-well white culture plate plate (Fisher Scientific® catalog #07-200-589) containing Steady-Glo® luciferase assay reagent (prepared according to the instructions in the above-identified kit). The sample was homogenized by quickly pipetting up and down several times, and luminescence was then promptly recorded at 25° C.

As illustrated by FIG. 14 and FIG. 15, the results of this experiment indicated that the ABPV viral IRES (also referred to as “GD-IRES-2.1”) displayed the highest expression level upon transfection. Four replicates for each Viral IRES Vector were evaluated using the agroinfiltration and luciferase assay protocols described above. Average expression level data is shown in FIG. 14, whereas the raw data for each replicate is shown in FIG. 15. IRES riboswitches for tobacco, as well as other agriculturally-significant crops such as corn, soybean, etc., can be developed using the principles described herein and the transfection and analysis techniques described in this example.

### REFERENCES

1. Green, A. A., Silver, P. A., Collins, J. J. & Yin, P. Toehold switches: De-novo-designed regulators of gene expression. Cell 159, 925-939 (2014).
2. Hanewich-Hollatz, M. H., Chen, Z., Hochrein, L. M., Huang, J. & Pierce, N. A. Conditional Guide RNAs: Programmable Conditional Regulation of CRISPR/Cas Function in Bacterial and Mammalian Cells via Dynamic RNA Nanotechnology. ACS Cent. Sci. 5, 1241-1249 (2019).
3. Siu, K. H. & Chen, W. Riboregulated toehold-gated gRNA for programmable CRISPR-Cas9 function. Nat. Chem. Biol. 15, 217-220 (2019).
4. Kim, J. et al. De novo-designed translation-repressing riboregulators for multi-input cellular logic. Nat. Chem. Biol. 15, 1173-1182 (2019).
5. Wang, S., Emery, N. J. & Liu, A. P. A Novel Synthetic Toehold Switch for MicroRNA Detection in Mammalian Cells. ACS Synth. Biol. 8, 1079-1088 (2019).
6. Litke, J. L. & Jaffrey, S. R. Highly efficient expression of circular RNA aptamers in cells using autocatalytic transcripts. Nat. Biotechnol. 37, 667-675 (2019).
7. Felletti, M., Stifel, J., Wurmthaler, L. A., Geiger, S. & Hartig, J. S. Twister ribozymes as highly versatile expression platforms for artificial riboswitches. Nat. Commun. 7, 2-9 (2016).
8. Zhong, G., Wang, H., Bailey, C. C., Gao, G. & Farzan, M. Rational design of aptazyme riboswitches for efficient control of gene expression in mammalian cells. Elife 5, (2016).
9. Zhong, G. et al. A reversible RNA on-switch that controls gene expression of AAV-delivered therapeutics in vivo. Nat. Biotechnol. doi:10.1038/s41587-019-0357-y
10. Ruehle, M. D. et al. A dynamic RNA loop in an IRES affects multiple steps of elongation factor-mediated translation initiation: (A) Schematic of the IGR IRES initiation factor-independent translation initiation mechanism. The IGR IRESs occupy the same binding sites as tRNAs i. Elife 4, 1-24 (2015).
11. Kanamori, Y. & Nakashima, N. A tertiary structure model of the internal ribosome entry site (IRES) for methionine-independent initiation of translation. Rna 7, 266-274 (2001).
12. Zhu, J. et al. Crystal structures of complexes containing domains from two viral internal ribosome entry site (IRES) RNAs bound to the 70S ribosome. Proc. Natl. Acad. Sci. U.S.A 108, 1839-1844 (2011).
13. Yamamoto, H., Nakashima, N., Ikeda, Y. & Uchiumi, T. Binding mode of the first aminoacyl-tRNA in translation initiation mediated by Plautia stali intestine virus internal ribosome entry site. J. Biol. Chem. 282, 7770-7776 (2007).
14. Colussi, T. M. et al. Initiation of translation in bacteria by a structured eukaryotic IRES RNA. Nature 519, 110-113 (2015).
15. Ogawa, A., Masuoka, H. & Ota, T. Artificial OFF-Riboswitches That Downregulate Internal Ribosome Entry without Hybridization Switches in a Eukaryotic Cell-Free Translation System. ACS Synth. Biol. 6, 1656-1662 (2017).
16. Hodgman, C. E. & Jewett, M. C. Characterizing IGR IRES-mediated translation initiation for use in yeast cell-free protein synthesis. N. Biotechnol. 31, 499-505 (2014).
17. Nishiyama, T. et al. Structural elements in the internal ribosome entry site of Plautia stali intestine virus responsible for binding with ribosomes. Nucleic Acids Res. 31, 2434-2442 (2003).
18. Wesselhoeft, R. A. et al. RNA Circularization Diminishes Immunogenicity and Can Extend Translation Duration In Vivo. Mol. Cell 74, 508-520.e4 (2019).
19. Sandig, V., Lieber, A., Bahring, S. & Strauss, M. A phage T7 class-III promoter functions as a polymerase II promoter in mammalian cells. Gene (1993). doi:10.1016/0378-1119(93)90302-J
20. Bensaude, O. Inhibiting eukaryotic transcription. Transcription 2, 103-108 (2011).
21. Keys, D. A. et al. Multiprotein transcription factor UAF interacts with the upstream element of the yeast RNA polymerase I promoter and forms a stable preinitiation complex. Genes Dev. 10, 887-903 (1996).
22. Vu, L., Siddiqi, I., Lee, B. S., Josaitis, C. A. & Nomura, M. RNA polymerase switch in transcription of yeast rDNA: Role of transcription factor UAF (upstream activation factor) in silencing rDNA transcription by RNA polymerase II. Proc. Natl. Acad. Sci. U.S.A 96, 4390-4395 (1999).
23. Bao, C. et al. mRNA stem-loops can pause the ribosome by hindering A-site tRNA binding. Elife 9, 1-67 (2020).
24. Hoffmann, E., Neumann, G., Hobom, G., Webster, R. G. & Kawaoka, Y. “Ambisense” approach for the generation of influenza A virus: vRNA and mRNA synthesis from one template. Virology 267, 310-317 (2000).
25. Czudai-Matwich, V., Schnare, M. & Pinkenburg, O. A simple and fast system for cloning influenza A virus gene segments into pHW2000- and pCAGGS-based vectors. Arch. Virol. 158, 2049-2058 (2013).
26. Yokoyama, T. et al. HCV IRES Captures an Actively Translating 80S Ribosome. Mol. Cell 74, 1205-1214.e8 (2019).
27. Ochs, K. et al. Interaction of Translation Initiation Factor eIF4B with the Poliovirus Internal Ribosome Entry Site. J. Virol. (2002). doi:10.1128/jvi.76.5.2113-2122.2002
28. Thompson, S. R. & Sarnow, P. Enterovirus 71 contains a type I IRES element that functions when eukaryotic initiation factor eIF4G is cleaved. Virology 315, 259-266 (2003).
29. Davila-Calderon, J. et al. IRES-targeting small molecule inhibits enterovirus 71 replication via allosteric stabilization of a ternary complex. Nat. Commun. 11, (2020).
30. Sadikoglou, E., Daoutsali, E., Petridou, E., Grigoriou, M. & Skavdis, G. Comparative analysis of internal ribosomal entry sites as molecular tools for bicistronic expression. J. Biotechnol. 181, 31-34 (2014).
31. McFadden, M. J. et al. A fluorescent cell-based system for imaging zika virus infection in real-time. Viruses 10, 13-18 (2018).
32. Medina, F. et al. Dengue virus: Isolation, propagation, quantification, and storage. Curr. Protoc. Microbiol. (2012). doi:10.1002/9780471729259.mc15d02s27
33. Murray, J. et al. Structural characterization of ribosome recruitment and translocation by type IV IRES. Elife (2016). doi:10.7554/eLife.13567
34. Zhao, E. M. et al. Optogenetic regulation of engineered cellular metabolism for microbial chemical production. Nature 555, 683-687 (2018).
35. Campeau, E. et al. A versatile viral system for expression and depletion of proteins in mammalian cells. PLoS One (2009). doi:10.1371/journal.pone.0006529
36. Bosch, I. et al. Serotype-specific detection of dengue viruses in a nonstructural protein 1-based enzyme-linked immunosorbent assay validated with a multi-national cohort. PLoS Negl. Trop. Dis. (2020). doi:10.1371/journal.pntd.0008203
37. Bosch, I. et al. Rapid antigen tests for dengue virus serotypes and zika virus in patient serum. Sci. Transl. Med. (2017). doi:10.1126/scitranslmed.aan1589

SEQUENCE LISTING
SEQ ID NO: 1 CrPV IRES
AAAAATGTGATCTTGCTTGTAAATACAATTTTGAGAGGTTAATAA
ATTACAAGTAGTGCTATTTTTGTATTTAGGTTAGCTATTTAGCTT
TACGTTCCAGGATGCCTAGTGGCAGCCCCACAATATCCAGGAAGC
CCTCTCTGCGGTTTTTCAGATTAGGTAGTCGAAAAACCTAAGAAA
TTTACCT
SEQ ID NO: 2 Os_PCD gene (coding sequence); DNA
ACGCGCTACCACACGCCAGGCAAACCCACACGCCGCCTTCCCGCC
GCCTCCGCTCTTCTTCCCCCAACCGCGTCGCAAGCCCACGGCGAG
CGCCGCCGCCGTCCCACCGTCGCCGATGACCCGCGGCGTCGCTAT
GGCGCACGCGAGGCTCCTCCTGGCGCGCTACTACGCGATGGCGGC
CCCCAGTTGGCCTACCGTATCCAAGAACCTTCCGCTGCTGGGCCA
TGGACGTTCTCATCATCCGATGTATGCAAGTCAAGACGAGATTAA
AATGTCAAGTAGAAGATGGTGCCATGGTTCTCCTGACAATCAAGA
GCTGGCAAAGAAGATTTGTGTTCCATGCAATTCTAAGGATATACA
TGCCATGCCAGAAGATTCTGCTAAGAAGATGCTAGAACAAGTGGG
TGGTTGGGAATTAGCAACCGAAGGTGACATTCTGAAATTGCACAG
AGCATGGAAGGTGAAGAATTTTGTAAAAGGGCTTGAATTCCTTCA
GCTTGTTGCTGCTGTTGCTGAGGAAGAAGGCCACCACCCTGATCT
TCATCTTGTCGGTTGGAACAATGTGAAAATTGATGTTTGGACTCA
CTCTGTCAGAGGCTTAACTGACAATGATTTCATCCTTGCTGCGAA
GATCAATAACCTCAACTTAGAAGGCCTCTTAAGCAAGAAAGCTAC
AGTCCAAAAGTAGTTCGCGCAGATGAAGCGATTGATTTCTGACGA
GATTTATGTACTTGCTTCGGTTAGAATGCGTGAGTTGAGGTATCT
TTAACTTGATAAGCTTTTCTTGAATTTGTACCGGGAGTTTCTCTG
TTCTTTCTAAATTTTACCAATGCTGTTGGACCGGGTCT
SEQ ID NO: 3 Os_Hsp70 (coding sequence); DNA
ATGGTGGAGGAGCTCGGCTTCGACATCAATGCCGGCAGAGAGCTT
GGCATAACAGCTTTGGCTTCTGCTGCGTTGGATGGAAGGCTGGCT
ACTGCAAGGTACCTTCTTGACCATGGGGCTGATCCAAATAAGAAG
TGCAATGCAGGCTCTGACAAGCAGGATGATCATAATAAAAAGGCT
CAGTTCAAACTACGTGGTGAGAAAGCTATTAAGGACAAGCATGAT
GAGCAGGATAAAAAGGCTCAGCTCAAACTACAGGGTGAGAAAGCT
GTTAAGAGAAAGGACTACCATGGCGCCTCAATTTTCTACACTGAG
GCAATTGAGCTGGATCCTACTGATGCAACATTGTATTCCAATAGG
AGCCTTTGCCATCTTCAAATGACCGAAGCTTTGTTTGATGCTGAT
TATTGCATAAAATCGCGGCCTGAATGGTTAAAAGGTTACTACAGG
AAAGGGGCTGCTCTCATGTTGCTGAAGGAGTATGAAAAGGCATGT
GACGCATTCTTGGCTGGACTGAAGTTGGATCCTTTGAATGCTGAG
ATGGAGAAAGTATTCCGGGAGGCGGTCGAGGCGATGAAGAAGCAT
CATGTTACTACAAAAAGCTTCAAGCCATCAGATTAG
SEQ ID NO: 4 APX promoter; DNA
GGTGAGGTGAGGTGAGTTGAGTTGGGGATTGATTGATTGATTCGG
ATTGGGAAGAAGAAGAAGCAGGGGAGCATGGGCAGCAAGTCGTAC
CCGACGGTGAGCGATGAGTACCTGGCGGCCGTGGGCAAGGCGAAG
CGCAAGCTCCGCGGCCTCATCGCCGAGAAGAACTGCGCCCCACTC
ATGCTCCGCCTCGCGTGGCACTCTGCTGGCACCTTCGATGTGTCG
TCGAGGACCGGCGGGCCCTTCGGCACCGTGAAGAACCCCGGCGAG
CAGTCCCACGCCGCCAACGCCGGCCTCGACATCGCCGTCAGGCTT
CTCGACCCCATCAAGGACCAACTTCCCATCCTCTCCTACGCCGAC
TTCTACCAGCTTGCTGGCGTTGTGGCCGTCGAGGTCACCGGCGGA
CCTGAGGTCCCCTTCCATCCGGGCAGGCAGGACAAGCCTGAGCCT
CCTCCTGAAGGCCGTCTTCCTGATGCCACACAAGGTTCTGACCAC
CTAAGGCAGGTCTTTTCTGCGCAGATGGGTTTGAGTGACAAGGAC
ATAGTTGCTCTTTCTGGTGGTCACACCCTGGGAAGATGCCACAAG
GAGAGATCTGGCTTTGAGGGAGCCTGGACGTCCATCCCTTTGATC
TTTGACAACTCTTACTTCACCGAGCTTGTGAGTGGCGAGAAGGAA
GGCCTTCTTCAGCTGCCAAGTGACAAAGCCCTCATGGCTGACCCA
GCCTTCCGTCCACTGGTGGAGAAATATGCTGCGGATGAGGACGCC
TTCTTTGCGGATTACGCCGAGGCCCACCTCAAGCTCTCTGAACTG
GGATTTGCTGAGGAATAAGAAGCCTTTAGAGAGCGGGATATCCGC
AAAAGATTAATGCCGATTTGTATTTTGCGCCTTAGAGTCAGTACG
ATCAAGACTGTCGTGGCGGTTGTAATAAAAATTAGTGTGCTTTGG
GCCATCTTTTTATGTGATTCCAATTGTCTTTCTCTTCATTCTTGC
TTTGATGCTCTTTGTCTGGACCTCTAGACCGCCGTATTGTACTGT
GGAGTTTCAAAGTTACCAAGCTATTTGCTGTCAAGATAACTATGG
ATTGAATTCCCCTTGATGGATGAACCAACTGTTGTTGTTTGCCCG
TTCTTCAGCTTTCGTTTGTGCGGCCATCGATCGCCATGCGTTGCT
TAAACCCATTTCTAGCTCCCCTACCCTGCTGCATCCGCCCTCTTC
TGCGCGATCGTTGGATTGCGAGTGGTTGGCTGGTTGCACGACTTG
TGGAGACCGAAACAAATAATTTTTGGTCAAATTGATCGGTGGTAC
TGTCGGAGCATCTATTTTTTCTTTAGCTTAGATCGTATAATTGTA
GGATTGGGATTTGTATATTAATATATACAGGTCGATTAAAACT
SEQ ID NO: 5 Os_PCD; amino acid sequence
MTRGVAMAHARLLLARYYAMAAPSWPTVSKNLPLLGHGRSHHPMY
ASQDEIKMSSRRWCHGSPDNQELAKKICVPCNSKDIHAMPEDSAK
KMLEQVGGWELATEGDILKLHRAWKVKNFVKGLEFLQLVAAVAEE
EGHHPDLHLVGWNNVKIDVWTHSVRGLTDNDFILAAKINNLNLEG
LLSKKATVQK
SEQ ID NO: 6 Gm_HSF (coding sequence); DNA
CCAACACTAATCTAAGCTCAAAACAAACACAACTTCCTAACGTAG
TCACAGAGTCAAAAACATTGTTTCACTTAAACACATTCGCACATA
AAAAGAAACCATTAACATTTAACCATACTACTAGATTATTAATTA
CTAGTACTTGTGTTGTTGTGTCACTCAAACTAAACTAAACTCAAT
TTCCAATTTCCTTAATCTTCTTGTGTTGATGTGCATGGAGAGAAT
CAGAGTGAAGGAGGAAGAAGCAATGACATGTGGAGGTGGTGGTTC
ATCATCTTCAAGCTTTATTTCTCCACAGGCACAGCCCATGGAAGG
GTTGCACGAGGTGGGTCCCCCTCCGTTTCTGAGCAAGATCTTTGA
CATGGTGGAAGATCCTTCCACGGATTCCATTGTTTCATGGAGCAT
GGCTCGCAACAGCTTTGTCGTGTGGGACTCTCACAAGTTTTCTGC
TCACATTCTGCCTCGTTACTTCAAGCACGCCAATTTCTCCAGCTT
CATTCGCCAGCTCAACACCTATGGGTTTAGGAAAGTTGATCCTGA
TAAATGGGAATTTGCAAACGAAGGTTTTCTGGCAGGACAAAGGCA
CTTATTGAAGACCATAAAGAGAAGAAGAAATGTGTCACACAGCAA
TCAACAAAAAGGAGGAAGTGGAGCTTGTGTTGAAGTGGGGAAGTT
TGGACTAGAAGGTGAGTTGGAGAGGTTGAAAAGGGATAGAAACAT
TTTGATGGCAGAAATTGTGAGATTGAGGCACCAACAACTGAACTC
AAGGGACCAATTAAGTGCCATGGAGGCAAGAATGCAAGCCACAGA
GAAGAAACAGCAACAAATGATGAGTTTCTTAGCAAAAGCACTAAG
CAATCCATCTTTCATGCAGCAATTAGTCCACAAAACCCCCCAAAG
CAGAGAAGTGTTGTTGGGTGTTGAAATCAATAGGAAAAGGAGACT
ACCTGCTTGTCCAAGTGTGGAGAACTTGCAACAAGATAATCAAGA
TTTGGCAACTATGGAGACTGATATGGACACTTTTTTCGCACCGGC
TTATGATAATGAATTCGGTAATGAAATCGATGAGCCTGCATCGAT
TTTGGTTGAGGATTCAATCTTGGAGGACTTTCTCAACAAAGACTT
GATTACTGGGAATCCGGAGGATGAAGTTATAATTGGTGATTGCAC
CCAAGTCGATGTGCCGATGGAGGATTTGGTAGCAAACCCTGATGA
TTGGAGTGAGCAATTGCAGGACCTTGTGGATCATATGGACTACCT
AGGATCCAAACCATAGAGGGATGAGAATTATACTATTATTGTGTC
CTCAGCTTGGTCTCTTTAATTTCATTCACATTGCATCATGATGGT
ACAATTTGGTACTTTTGTTACTACTTTTGGTTTTGGTTTCAATTT
TATGTGATCTTAATGATAAGTGTGAATTGTTTTTATGCAGCAGGG
AAGGTGGTAGATACAGAAGAAGCAAACAAATACTGCTCTTGTTTG
GATCCTTTATGAAGTTTTATGAATATTACTTGTGCTAATGCTACA
GATCATTTTATTAATTATTTGGATGACCTATTTGTTTGCATTTTA
TGTTATGCAGGAGAGACAAGTGGGGGCACTTCATGGTTGTGATCC
AAATAATTGCTGAGAAATTGTAATGGTATCATTCTGCTTCATAGG
ATATCATTGGCTGGATTTCATTGTTCAGGAGAGGATATAAAAACT
ATCATCTAGTTCTTCTAGTACGAATAGCCTTTCAGAGGAACCACA
TTTGTTCCCATTCTTTTTCCTTCAAAAAATTGGATATTGTTGGTG
CTA
SEQ ID NO: 7 tomato SP5G (genomic sequence); DNA
AGGTGCATTCCTTCTTATAATTTTTTCACCTTCATTATTATTAAT
TAAAAGTCCCAATAAACAAAGGAAAAAGTTCTATCACTTTTTTAC
AATTGGAAAAAAGAGATTCTTTTAAGGAATGTGTGGTGACAAATA
AGCATCCTATTTTCTTCTGTTACTAAAGCCTCTAAAAAAATAAAA
AATAAAAATATATATACTTAAACTCACAAGTTTAGGGCAATTTTG
ATGCATCTTTATTTTGTTTATCAAACTCTATATATAGTCATTCAT
AAAAATGATAGTACGACACATAAATCACTCCATATTTTATTATAT
TGTAATTATGATTCGAGAAAGGATTTAATTTCTCAAAATATAATA
TAAATAGACTATCGTAATATAAATATTAGTACTATCTAATGAAGT
ACGATTTGATGAAGTGTAAAGTTAACTTACATATGACCTAGAGAA
ACCACTTAAGTAGCAATGAATAATCCAAGATATATATATACTTAA
ACTCACAAGTTACATTTTTGACGTATCTTTATTTTGTTTATTAAA
CTCTATATATAGTCATTCATAAAAATGATAATATGACGCATAAAT
CATTCCATATTTTATTAAATTATAATTAAGATTTGAAAAAAGAAT
TTACTTCTTCAAAATATAATCTAAATAGTCTATCGTAATATAAAT
ATTAATGTTATCTAGTGAAGTATGATTTAATGAAGTGTAAAGTTA
ACTTACATATGACCTAGAGAAATCACTTAAGTAGCGATGAATAAT
CCAAGAATATGTTTGGTCCTTTATTCTTTCTTGTCATGGCTCATG
TATCCATGCACTTTATTATAACAATTCGAGAAGTGTTATAATTAT
GGTGATTCTCTTATTTAAAATTTTTTGAACTATTAATAAAGTAAA
TGAATAATAATAATAATAATATGAATTAGGAAAATATTTATGTAT
ATAATTTTTATGTCAAAATTACTTGATTCTCCACTTTACAGCTCA
ACAATTAACATATATGGTTTCCCCTTAAAGAAAAACTTCAAAAAG
ATTCCTATGATGGTAAAGAAACGTTTGGCCATAAAAATTAAATAT
TTTTCAATTTTCAAATCGAAATTTTTTTGATCACTATGGATCTGA
TATAAACAGTCTCCCTATTACGAAAAAGTAAGAGTAAGGTCTGCA
TACATCTTATCCTCTTTAAACCTCACAGTTTGAAGATGCGACTTT
GTTTGATTATACTTTTTCCAAAGGAGAGAATTAAGAGATTATATT
TGGAATTACGCAGACAAAATTTGAAAGACATCTTATAAGTTTGAA
ATCCAATTACAAGTGGAATTTAAAATTTTCACGACTTGTCAACCA
TTGATTCTCAAATAAAGTGAAAAATTATTCCAAAAACAAATAAAT
ATTTTTTTTATGACCAAATATGTCCTCAAGAACATATGAAAGCTC
TCTAGTCATGAGTATAAATAACAAGGGCTAGCTAGCTCTTGTCTA
CTCATAAAATATCATCCATCCATCTCATGTAATAAACAAAAATTG
AGCTTATTAATTATAATTGAGAAGAAAAAAAATCATGCCTAGAGA
TCCTTTAATAGTTTCTGGAGTTGTTGGAGATGTTGTTGATCCATT
CACAAGATGTGTAGACTTTGGTGTGGTTTACAACAATAGGGTGGT
CTATAATGGATGTTCCTTGAGGCCTTCACAAGTTGTCAATCAACC
TAGGGTTGACATTGATGGAGACGATCTTCGTACTTTTTACACTCT
GGTATAAACTCATCGTTTTATTTCATATGATATACATATATATAT
ATATATATATATATATATATATATATATTTTCTTTCTATTTATAC
ATTTTAATATCTCTAAATTATTAACCTTTTGTCAATTGATTATGA
GTAGAAGATCAAAAGGACAATATGTGCAAAGGCTTCTAATTATGT
GAATTTGTGTTAGTTTTAATTTTGATTCACCATCTAAGTACTTGT
TTTGTGGTTTTTATTTGAATTTGAGAACTCATAACATACTATTTA
TGATAATAAAAAATGTTAGTAACATGTATGTTTAATATTGCAAGC
TTGAAAATATACAATATTTTTAAATTACTAATAATGTCATGTAAT
ACATTTGGATATACAATATGGAAAATTATTTTTCCTAATTTTCAA
AATATTTGAAATGTTTCTTTTCTTTTTGGAAGATTATGGTGGATC
CTGATGCTCCAAACCCTAGCAACCCAAACCTGAGGGAATATTTGC
ACTGGTAAGTCATCTAGCTTATATTATATATATATATATATATAT
ATATATATATTATATAAATAGATAAAAATATTCATTTTGTTATAT
ACTTCTTATTTCTCTTAAATCAATCGTCGATAGCGAAGACAAAAA
TGTATGTGAGATTATATAAGAACCTAAGGAAAGTATTATTTCATA
AAATGATAACTTTCTGATACACAAATTAATCAATATTTCAAATAA
ATACCAAATATCGAATAACAACGTAAAAAAATAATAACTATTATC
GATTGCTTAATCCCCTTACAATTAATGTACCTAAACCTCTTTTTT
TTTTTAAAAAAAAAATAATAATAATAATGTTTAACACATTATTTT
TTTAATAGGTTGGTCACAGATATCCCAGCAGCCACAGGAGCAACC
TTTGGTAAGTTTTTCTTACATTATTACCTAATGGCTCGTAATTAC
GCAGTGACGAAGCAAGAAATTTAAATATACTTTATATTTACGATA
CATTGTATCCGTATCACTACATTTTTAATATAAGACGGTTAGTAA
TATACAAAATACAACTTGTATCATCATCACCTTAGTAGTACATTA
TTAGTACTATAGGCCCAATTATGACTACTAATAAAATAAGACTTA
AAAAGAAACATAAAATCAAAATGAAGTATATACTATGTATATAAA
TGTTTTTGAAACAAGGAAAATACGCGTATTGAATGTCTTTGTTAC
TAAACTCAAACTCTCGTTATACAGGCAATGAAGTCGTGGGCTACG
AGAGCCCACGACCCTCAATGGGAATCCATCGTTATATTTTCGTGT
TGTATCGACAATTGGGCTGCGATGCCATCGATGCACCGGACATAA
TCGATTCTAGACAAAATTTCAACACAAGAGACTTTGCTAGGTTTC
ACAATCTAGGTTTGCCTGTTGCTGCTGTTTACTTCAATTGCAATA
GGGAAGGTGGTACCGGTGGTCGTCGCCTATAAATCACCCCCCTCT
CCTCGGGGTGCGATCCGTTCTCGAACTCTGTGTCAATGTCAGATG
TTTTGTGTAACGGATTTTTTGTTTGATAGTCACTCAGCTAAATTG
CTTATTAACTCAGAAAGTCATTTTTCTTTTTATCGAAGAAAATTG
AAATTATGAGATAATAGCTATTATAGTTGAGTGATAATCTGAAAA
AAAAAATCAACCATAATATATATTATATATTATATATATACCCAA
AAAATAAAAGTCT
SEQ ID NO: 8 tomato SP5G; amino acid sequence
MPRDPLIVSGVVGDVVDPFTRCVDFGVVYNNRVVYNGCSLRPSQV
VNQPRVDIDGDDLRTFYTLIMVDPDAPNPSNPNLREYLHWLVTDI
PAATGATFGNEVVGYESPRPSMGIHRYIFVLYRQLGCDAIDAPDI
IDSRONFNTRDFARFHNLGLPVAAVYENCNREGGTGGRRL
SEQ ID NO: 9 UAF2 sequence
GGCAGCAGAGAGACCTGAAAAAGCAATTTTTCTGGAATTTCAGCT
GTTTCCAAACTCAATAAGTATCTTCTAGCAAGAGGGAATAGGTGG
GAAAAAAAAAAAGAGATTTC
SEQ ID NO: 10 Stop codon and stem loop sequence
CGTTGACTTGCGCAAGTCAACGTTAACACCGTTTTCGGGGGGTGA
CGATCGTCACCCCAATGGGTAAGGGTTCCatcgccgggaaacagc
tggagctTAGTAATGAATAAATGAACTGGACTGACTAATGTAGTC
AGTCCCTCGATCGATGCATGCataccgtaaaggagaagctggtgg
aacgtgggatctctgtgcaaagctacaatggagatctattgtatg
aaccgtgggaATATCGGCCGGCCACGCgacagtgcgctcgggtgg
tccatccactcgacacacccgccaCCGGCAGCCGCTACCCGCGCG
CGGCCGGTATATTCTGGCCTTCCCACAAGGGAAGGCCAGGATCGT
CGATGCCGTGGCCAAGGGGTCATTGAGATAGCCTTTCATGATTTC
CGTCTGCGCGCGCAGACGGGCAAGGTAGAAGACCTTGGCTGTACC
ATTTTTTAACGGAGGAGTTACTTTCTAAGTATCATTTAGATTTGA
AC
SEQ ID NO: 11: CrPV toehold 8-6
AGTACCCTTCACCAAAGCAAAAATGTGATCTTGCTTGTAAATACA
ATTTTGAGAGGTTAATAAATTACAAGTAGTGCTATTTTTGTATTT
AGGTTAGCTATTTAGCTTTACGTTCCAGGATGCCTAGTGGCAGCC
CCACAATATCCAGGAAGXCCCTCTCTGCGGTTTTTCAGATTAGGT
AGTCGAAAAACCTAAGAAATTTACCTGCTAXCATTTCAAGATACC
Each “X” indicates a site for insertion.
SEQ ID NO: 12 CrPV toehold 6-7
AGTACCCTTCACCAAAGCAAAAATGTGATCTTGCTTGTAAATACA
ATTTTGAGAGGTTAATAAATTACAAGTAGTGCTATTTTTGTATTT
AGGTTAGCTATTTAGCTTTACGTTCCAGGATGCCTAGTGGCAGCC
CCACAATATCCAGGAAXGCCCTCTCTGXCGGTTTTTCAGATTAGG
TAGTCGAAAAACCTAAGAAATTTACCTGCTACATTTCAAGATACC
Each “X” indicates a site for insertion.
SEQ ID NO: 13 ABPV toehold 8-6
gattcctaaattactacttgtaatttgggaatcgcaacacaacat
ggttacccatagattgaggaaatttccaataaactcagtattaag
gcttgttgtgttggacaaggtgccctatttagggtgaggagcctt
actggcagccccagtgaatcctcXcattggataggaacagctata
ttgggtagttgtagcagttgtattcaaatgaatgcagcgttccga
aatatcatacctgccgatcXaagaaacaaata
Each “X” indicates a site for insertion.
SEQ ID NO: 14 HCV toehold 2-12
gccagccccctgatgggggcGACACTCCACCATGAATCACTCCcc
tgtgaggaactactgtcttcacgcagaaagcgtctagXccatggc
gttagtatgagtgtcgtgcagcctccaggaccccccctcccggga
gagccatagtggtctgcggaaccggtgagtacaccggaattgcca
ggacgaccgggtcctttcttggataaacccgctcaatgcctggag
atttgggcgtgcccccgcaagactgctagccgagtagtgttgggt
cgcgaaaggcctXtgtggtactgcctgatagggtgcttgcgagtg
ccccgggaggtctcgtagaccgtgcaccatgagcacgaatcctaa
acctcaaagaaaaacc
Each “X” indicates a site for insertion.
SEQ ID NO: 15 PV toehold 1-2
TTAAAACAGCTCTGGGGTTGTACCCACCCCAGAGGCCCACGTGGC
GGCTAGTACTCCGGTATTGCGGTACCCTTGTACGCCTGTTTTATA
CTCCCTTCCCGTAACTTAGACGCACAXAACCAAGTTCAATAGAAG
GGGGTACAAACCAGTACCACCACGAACAAGCACXTTCTGTTTCCC
CGGTGATGTCGTATAGACTGCTTGCGTGGTTGAAAGCGACGGATC
CGTTATCCGCTTATGTACTTCGAGAAGCCCAGTACCACCTCGGAA
TCTTCGATGCGTTGCGCTCAGCACTCAACCCCAGAGTGTAGCTTA
GGCTGATGAGTCTGGACATCCCTCACCGGTGACGGTGGTCCAGGC
TGCGTTGGCGGCCTACCTATGGCTAACGCCATGGGACGCTAGTTG
TGAACAAGGTGTGAAGAGCCTATTGAGCTACATAAGAATCCTCCG
GCCCCTGAATGCGGCTAATCCCAACCTCGGAGCAGGTGGTCACAA
ACCAGTGATTGGCCTGTCGTAACGCGCAAGTCCGTGGCGGAACCG
ACTACTTTGGGTGTCCGTGTTTCCTTTTATTTTATTGTGGCTGCT
TATGGTGACAATCACAGATTGTTATCATAAAGCGAATTGGATTGG
CCATCCGGTGAAAGTGAGACTCATTATCTATCTGTTTGCTGGATC
CGCTCCATTGAGTGTGTTTACTCTAAGTACAATTTCAACAGTTAT
TTCAATCAGACAATTGTATCATATG
Each “X” indicates a site for insertion.
SEQ ID NO: 16 EV71 mutant toehold 0-1
cacccactcacagggcccactgggcgctagcactctgattttacg
aaatccttgtgcgcctgttttatatcccttccXctaattcgaaac
gtagaagcaatgcgcaccactXgatcaatagtaggcgtaacgcgc
cagttacgtcatgatcaagcatatctgttcccccggactgagtat
caatagactgcttacgcggttgaaggagaaaacgttcgttatccg
gctaactacttcgagaagcccagtaacaccatggaagctgcaggg
tgtttcgctcagcacttcccccgtgtagatcaggtcgatgagcca
ctgcaatccccacaggtgactgtggcagtggctgcgttggcggcc
tgcctatggggagacccataggacgctctaatgtggacatggtgc
gaagagcctattgagctagttagtagtcctccggcccctgaatgc
ggctaatcctaactgcggagcacatgccttcaacccagagggtag
tgtgtcgtaacgggcaactctgcagcggaaccgactactttgggt
gtccgtgtttctttttatttttatattggctgcttatggtgacaa
ttacagaattgttaccatatagctattggattggccatccggtgt
gtaatagagctgttatatacctatttgttggctttgtaccactaa
ctttaaaatctataactaccctcaactttgtattaactctcaata
cagctgatcatg
Each “X” indicates a site for insertion.
SEQ ID NO: 17 PV toehold 0-5
xTTAAAACAGCTCTGGGGTTGTACCCACCCCAGAGGCCCACGTGG
CGGCTAGTACTCCGGTATTGCGGTACCCTTGTACGCCTGTTTTAT
ACTCCCTTCCCGTAACTTAGACGCACAAAACCAAGTTCAATAGAA
GGGGGTACAAACCAGTACCACCACGAACAAGCACTTCTGTTTCCC
CGGTGATGTCGTATAGACTGCTTGCGTGGTTGAAAGCGACGGATC
CGTTATCCGCTTATGTACTTCGAGAAGCCCAGTACCACCTCGGAA
TCTTCGATGCGTTGCGCTCAGCACTCAACCCCAGAGTGTAGCTTA
GGCTGATGAGTCTGGACATCCCTCACCGGTGACGGTGGTCCAGGC
TGCGTTGGCGGCCTACCTATGGCTAACGCCATGGGACGCTAGTIG
TGAACAAGGTGTGAAGAGCCTATTGAGCTACATAAGAATCCTCCG
GCCCCTGAATGCGGCTAATCCCAACCTCGGAGCAGGTGGTCACAA
ACCAGTGATTGGCCTGTCGTAACGCGCAAGTCCGTGGCGGAACCG
ACTACTTTGGGTGTCCGTGTTTCCTTTTATTTTATTGTGGCTGCT
TATGGTGACAATCACAGATTGTTATCATAAAGCGAATTGGATTGG
CCATxCCGGTGAAAGTGAGACTCATTATCTATCTGTTTGCTGGAT
CCGCTCCATTGAGTGTGTTTACTCTAAGTACAATTTCAACAGTTA
TTTCAATCAGACAATTGTATCATATG
Each “X” indicates a site for insertion.

1. A recombinant plant, comprising:
a) DNA encoding an mRNA which comprises a recombinant nucleic acid molecule, wherein the recombinant nucleic acid molecule comprises:
i) a first segment encoding a recombinant Group 1 Dicistroviridae internal ribosome entry site (IRES) that has been modified to incorporate exogenous nucleotide sequences at a first site and a second site, and
ii) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence; and
wherein the Group 1 Dicistroviridae IRES is configured to activate expression of the protein in response to the presence of an mRNA which comprises a segment that is the reverse complement of the first nucleotide sequence.

2. The recombinant plant of claim 1, wherein the mRNA was generated:
a) by a bacterial, viral, or fungal pathogen;
b) by an insect pest; or
c) by the recombinant plant, in response to an environmental stress.

3. The recombinant plant of claim 1, wherein the protein is:
a) capable of modulating a defense responsive to the bacterial, viral, or fungal pathogen, or to the insect pest. or
b) capable of modulating a metabolic pathway response to the environmental stress.

4. The recombinant plant of claim 1, wherein the plant is a soybean, tobacco, corn, canola, cotton, wheat, or rice plant.

5. The recombinant plant of claim 1, wherein the DNA is integrated into the genomic DNA of the plant, or a vector present in the plant.

6. The recombinant plant of claim 1, wherein the second nucleotide sequence is the reverse complement of substantially all of the first nucleotide sequence.

7. The recombinant plant of claim 1, wherein the Group 1 Dicistroviridae IRES is a cricket paralysis virus (CrPV), a Kashmir bee virus (KBV), an acute bee paralysis virus (ABPV), or a Plauta stali Intestine Virus (PSIV) IRES.

8. The recombinant plant of claim 1, wherein the first and second sites are each independently selected from any of Site 1, Site 2, Site 3, Site 4, Site 5, Site 6, Site 7, and Site 8.

9. The recombinant plant of claim 8, wherein the first and second sites respectively comprise: Site 1 and Site 2, Site 1 and Site 4, Site 1 and Site 5, Site 1 and Site 6, Site 1 and Site 7, Site 1 and Site 8, Site 2 and Site 6, Site 2 and Site 7, Site 4 and Site 6, Site 5 and Site 6, Site 5 and Site 7, Site 6 and Site 7, Site 8 and Site 2, Site 8 and Site 6, or Site 8 and Site 7.

10. The recombinant plant of claim 1, wherein the first nucleotide sequence is 25-80 nt in length.

11. The recombinant plant of claim 1, wherein the second nucleotide sequence is 8-25 nt in length.

12. The recombinant plant of claim 1, wherein the protein is a hormone or growth factor that modulates growth of the plant.

13. The recombinant plant of claim 1, wherein the protein is a heat shock protein.

14. The recombinant plant of claim 1, wherein the protein is a pesticide.

15. The recombinant plant of claim 1, wherein the activated expression of the protein comprises an expression level that is at least 1.5, 2.0, 2.5, 3.0, 3.5, 4.0, 4.0, or 5-fold higher than an endogenous expression level of the protein in at least one cell type or tissue in the plant.

16. A vector, comprising:
a) DNA encoding an mRNA which comprises a recombinant nucleic acid molecule, wherein the recombinant nucleic acid molecule comprises:
i) a first segment encoding a recombinant Group 1 Dicistroviridae internal ribosome entry site (IRES) that has been modified to incorporate exogenous nucleotide sequences at a first site and a second site, and
ii) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence; and
wherein the Group 1 Dicistroviridae IRES is configured to activate expression of the protein in response to the presence of an mRNA which comprises a segment that is the reverse complement of the first nucleotide sequence wherein the protein is capable of:
a) improving a plant's resistance to a disease;
b) modulating the plant's response to an insect, viral, bacterial or fungal pest; and/or
c) improving the rate of growth or development of the plant.

17. The vector of claim 16, wherein the plasmid further comprises an origin of replication configured to provide stable maintenance of the plasmid in an Agrobacterium cell.

18. The vector of claim 16, wherein the protein is a hormone or growth factor that modulates growth of the plant.

19. The vector of claim 16, wherein the protein is a heat shock protein.

20. The vector of claim 16, wherein the protein is a pesticide.

21. A recombinant plant cell comprising the vector of claim 16.

22. A method of generating a recombinant plant with improved resistance to a disease, a pest, or an environmental stress, comprising the steps of:
a) providing a recombinant DNA molecule that encodes an mRNA which comprises:
i) a first segment encoding a Group 1 Dicistroviridae internal ribosome entry site (IRES) that has been modified to incorporate exogenous nucleotide sequences at a first site and a second site, and
ii) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence; and wherein the Group 1 Dicistroviridae IRES is configured to activate expression of the protein in response to the presence of an mRNA which comprises a segment that is the reverse complement of the first nucleotide sequence.
b) introducing the recombinant DNA molecule into the plant.

23. The method of claim 22, where the protein is capable of:
a) improving the plant's resistance to disease;
b) modulating the plant's response to an insect, viral, bacterial or fungal pest; and/or
c) improving the rate of growth or development of the plant.

24. A recombinant nucleic acid molecule, comprising:
a) a first segment encoding a recombinant Group 1 Dicistroviridae internal ribosome entry site (IRES) that has been modified to incorporate exogenous nucleotide sequences at a first site and a second site, and
b) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence
wherein the first and second nucleotide sequences are capable of hybridizing when expressed in a plant cell under in vivo or in vitro conditions, causing the Group 1 Dicistroviridae IRES to fold into an inactivated state.
wherein the protein is capable of:
a) improving a plant's resistance to a disease;
b) modulating the plant's response to an insect, viral, bacterial or fungal pest; and/or
c) improving the rate of growth or development of the plant.

25. The recombinant nucleic acid molecule of claim 24, wherein the Group 1 Dicistroviridae IRES is further configured to fold into an activated state in the presence of a trigger RNA molecule comprising a third nucleotide sequence, wherein the third nucleotide sequence is the reverse compliment of the first nucleotide sequence of the recombinant nucleic acid molecule.

26. The recombinant nucleic acid molecule of claim 25, wherein the first nucleotide sequence is capable of hybridizing to the third nucleotide sequence when expressed in a plant cell under in vivo conditions, causing the Group 1 Dicistroviridae IRES to fold into the activated state.

27. A plasmid encoding the recombinant nucleic acid molecule of claim 24.

28. A plant cell comprising DNA encoding the recombinant nucleic acid molecule of claim 24, wherein the DNA is:
a) integrated into the genomic DNA of the plant cell, or
b) present on a plasmid or viral vector present within the plant cell.

29. A kit, comprising:
a) the plasmid of claim 27; and
b) a trigger RNA molecule comprising a third nucleotide sequence, wherein the third nucleotide sequence is the reverse compliment of the first nucleotide sequence of the recombinant nucleic acid molecule.

30. A method of activating and/or modulating expression of a protein, comprising:
a) providing a plant cell engineered to express the recombinant nucleic acid molecule of claim 24; and
b) introducing a trigger RNA molecule comprising a third nucleotide sequence into the plant cell, wherein the third nucleotide sequence is the reverse compliment of the first nucleotide sequence of the recombinant nucleic acid molecule;
wherein the first nucleotide sequence hybridizes to the third nucleotide sequence under in vivo conditions, causing the Group 1 Dicistroviridae IRES to fold into an activated state.

31. The method of claim 30, wherein the plant cell engineered to express the recombinant nucleic acid molecule is provided by introducing the recombinant nucleic acid molecule into the plant cell.

32. A method for controlling cell differentiation, comprising
a) providing a plant cell engineered to express the recombinant nucleic acid molecule of claim 24; and
b) culturing the plant cell;
wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of the engineered plant cell.


SEQUENCE LISTING (17 sequences)

SEQ ID NO: 1 - DNA, 187 bp, Artificial Sequence
  Synthetic polynucleotide
aaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 2 - DNA, 848 bp, Artificial Sequence
  Synthetic polynucleotide
acgcgctaccacacgccaggcaaacccacacgccgccttcccgccgcctccgctcttcttcccccaaccgcgtcgcaagcccacggcgagcgccgccgccgtcccaccgtcgccgatgacccgcggcgtcgctatggcgcacgcgaggctcctcctggcgcgctactacgcgatggcggcccccagttggcctaccgtatccaagaaccttccgctgctgggccatggacgttctcatcatccgatgtatgcaagtcaagacgagattaaaatgtcaagtagaagatggtgccatggttctcctgacaatcaagagctggcaaagaagatttgtgttccatgcaattctaaggatatacatgccatgccagaagattctgctaagaagatgctagaacaagtgggtggttgggaattagcaaccgaaggtgacattctgaaattgcacagagcatggaaggtgaagaattttgtaaaagggcttgaattccttcagcttgttgctgctgttgctgaggaagaaggccaccaccctgatcttcatcttgtcggttggaacaatgtgaaaattgatgtttggactcactctgtcagaggcttaactgacaatgatttcatccttgctgcgaagatcaataacctcaacttagaaggcctcttaagcaagaaagctacagtccaaaagtagttcgcgcagatgaagcgattgatttctgacgagatttatgtacttgcttcggttagaatgcgtgagttgaggtatctttaacttgataagcttttcttgaatttgtaccgggagtttctctgttctttctaaattttaccaatgctgttggaccgggtct

SEQ ID NO: 3 - DNA, 621 bp, Artificial Sequence
  Synthetic polynucleotide
atggtggaggagctcggcttcgacatcaatgccggcagagagcttggcataacagctttggcttctgctgcgttggatggaaggctggctactgcaaggtaccttcttgaccatggggctgatccaaataagaagtgcaatgcaggctctgacaagcaggatgatcataataaaaaggctcagttcaaactacgtggtgagaaagctattaaggacaagcatgatgagcaggataaaaaggctcagctcaaactacagggtgagaaagctgttaagagaaaggactaccatggcgcctcaattttctacactgaggcaattgagctggatcctactgatgcaacattgtattccaataggagcctttgccatcttcaaatgaccgaagctttgtttgatgctgattattgcataaaatcgcggcctgaatggttaaaaggttactacaggaaaggggctgctctcatgttgctgaaggagtatgaaaaggcatgtgacgcattcttggctggactgaagttggatcctttgaatgctgagatggagaaagtattccgggaggcggtcgaggcgatgaagaagcatcatgttactacaaaaagcttcaagccatcagattag

SEQ ID NO: 4 - DNA, 1393 bp, Artificial Sequence
  Synthetic polynucleotide
ggtgaggtgaggtgagttgagttggggattgattgattgattcggattgggaagaagaagaagcaggggagcatgggcagcaagtcgtacccgacggtgagcgatgagtacctggcggccgtgggcaaggcgaagcgcaagctccgcggcctcatcgccgagaagaactgcgccccactcatgctccgcctcgcgtggcactctgctggcaccttcgatgtgtcgtcgaggaccggcgggcccttcggcaccgtgaagaaccccggcgagcagtcccacgccgccaacgccggcctcgacatcgccgtcaggcttctcgaccccatcaaggaccaacttcccatcctctcctacgccgacttctaccagcttgctggcgttgtggccgtcgaggtcaccggcggacctgaggtccccttccatccgggcaggcaggacaagcctgagcctcctcctgaaggccgtcttcctgatgccacacaaggttctgaccacctaaggcaggtcttttctgcgcagatgggtttgagtgacaaggacatagttgctctttctggtggtcacaccctgggaagatgccacaaggagagatctggctttgagggagcctggacgtccatccctttgatctttgacaactcttacttcaccgagcttgtgagtggcgagaaggaaggccttcttcagctgccaagtgacaaagccctcatggctgacccagccttccgtccactggtggagaaatatgctgcggatgaggacgccttctttgcggattacgccgaggcccacctcaagctctctgaactgggatttgctgaggaataagaagcctttagagagcgggatatccgcaaaagattaatgccgatttgtattttgcgccttagagtcagtacgatcaagactgtcgtggcggttgtaataaaaattagtgtgctttgggccatctttttatgtgattccaattgtctttctcttcattcttgctttgatgctctttgtctggacctctagaccgccgtattgtactgtggagtttcaaagttaccaagctatttgctgtcaagataactatggattgaattccccttgatggatgaaccaactgttgttgtttgcccgttcttcagctttcgtttgtgcggccatcgatcgccatgcgttgcttaaacccatttctagctcccctaccctgctgcatccgccctcttctgcgcgatcgttggattgcgagtggttggctggttgcacgacttgtggagaccgaaacaaataatttttggtcaaattgatcggtggtactgtcggagcatctattttttctttagcttagatcgtataattgtaggattgggatttgtatattaatatatacaggtcgattaaaact

SEQ ID NO: 5 - protein, 190 aa, Artificial Sequence
  Synthetic polynucleotide
MTRGVAMAHARLLLARYYAMAAPSWPTVSKNLPLLGHGRSHHPMYASQDEIKMSSRRWCHGSPDNQELAKKICVPCNSKDIHAMPEDSAKKMLEQVGGWELATEGDILKLHRAWKVKNFVKGLEFLQLVAAVAEEEGHHPDLHLVGWNNVKIDVWTHSVRGLTDNDFILAAKINNLNLEGLLSKKATVQK

SEQ ID NO: 6 - DNA, 1803 bp, Artificial Sequence
  Synthetic polynucleotide
ccaacactaatctaagctcaaaacaaacacaacttcctaacgtagtcacagagtcaaaaacattgtttcacttaaacacattcgcacataaaaagaaaccattaacatttaaccatactactagattattaattactagtacttgtgttgttgtgtcactcaaactaaactaaactcaatttccaatttccttaatcttcttgtgttgatgtgcatggagagaatcagagtgaaggaggaagaagcaatgacatgtggaggtggtggttcatcatcttcaagctttatttctccacaggcacagcccatggaagggttgcacgaggtgggtccccctccgtttctgagcaagatctttgacatggtggaagatccttccacggattccattgtttcatggagcatggctcgcaacagctttgtcgtgtgggactctcacaagttttctgctcacattctgcctcgttacttcaagcacgccaatttctccagcttcattcgccagctcaacacctatgggtttaggaaagttgatcctgataaatgggaatttgcaaacgaaggttttctggcaggacaaaggcacttattgaagaccataaagagaagaagaaatgtgtcacacagcaatcaacaaaaaggaggaagtggagcttgtgttgaagtggggaagtttggactagaaggtgagttggagaggttgaaaagggatagaaacattttgatggcagaaattgtgagattgaggcaccaacaactgaactcaagggaccaattaagtgccatggaggcaagaatgcaagccacagagaagaaacagcaacaaatgatgagtttcttagcaaaagcactaagcaatccatctttcatgcagcaattagtccacaaaaccccccaaagcagagaagtgttgttgggtgttgaaatcaataggaaaaggagactacctgcttgtccaagtgtggagaacttgcaacaagataatcaagatttggcaactatggagactgatatggacacttttttcgcaccggcttatgataatgaattcggtaatgaaatcgatgagcctgcatcgattttggttgaggattcaatcttggaggactttctcaacaaagacttgattactgggaatccggaggatgaagttataattggtgattgcacccaagtcgatgtgccgatggaggatttggtagcaaaccctgatgattggagtgagcaattgcaggaccttgtggatcatatggactacctaggatccaaaccatagagggatgagaattatactattattgtgtcctcagcttggtctctttaatttcattcacattgcatcatgatggtacaatttggtacttttgttactacttttggttttggtttcaattttatgtgatcttaatgataagtgtgaattgtttttatgcagcagggaaggtggtagatacagaagaagcaaacaaatactgctcttgtttggatcctttatgaagttttatgaatattacttgtgctaatgctacagatcattttattaattatttggatgacctatttgtttgcattttatgttatgcaggagagacaagtgggggcacttcatggttgtgatccaaataattgctgagaaattgtaatggtatcattctgcttcataggatatcattggctggatttcattgttcaggagaggatataaaaactatcatctagttcttctagtacgaatagcctttcagaggaaccacatttgttcccattctttttccttcaaaaaattggatattgttggtgcta

SEQ ID NO: 7 - DNA, 3478 bp, Artificial Sequence
  Synthetic polynucleotide
aggtgcattccttcttataattttttcaccttcattattattaattaaaagtcccaataaacaaaggaaaaagttctatcacttttttacaattggaaaaaagagattcttttaaggaatgtgtggtgacaaataagcatcctattttcttctgttactaaagcctctaaaaaaataaaaaataaaaatatatatacttaaactcacaagtttagggcaattttgatgcatctttattttgtttatcaaactctatatatagtcattcataaaaatgatagtacgacacataaatcactccatattttattatattgtaattatgattcgagaaaggatttaatttctcaaaatataatataaatagactatcgtaatataaatattagtactatctaatgaagtacgatttgatgaagtgtaaagttaacttacatatgacctagagaaaccacttaagtagcaatgaataatccaagatatatatatacttaaactcacaagttacatttttgacgtatctttattttgtttattaaactctatatatagtcattcataaaaatgataatatgacgcataaatcattccatattttattaaattataattaagatttgaaaaaagaatttacttcttcaaaatataatctaaatagtctatcgtaatataaatattaatgttatctagtgaagtatgatttaatgaagtgtaaagttaacttacatatgacctagagaaatcacttaagtagcgatgaataatccaagaatatgtttggtcctttattctttcttgtcatggctcatgtatccatgcactttattataacaattcgagaagtgttataattatggtgattctcttatttaaaattttttgaactattaataaagtaaatgaataataataataataatatgaattaggaaaatatttatgtatataatttttatgtcaaaattacttgattctccactttacagctcaacaattaacatatatggtttccccttaaagaaaaacttcaaaaagattcctatgatggtaaagaaacgtttggccataaaaattaaatatttttcaattttcaaatcgaaatttttttgatcactatggatctgatataaacagtctccctattacgaaaaagtaagagtaaggtctgcatacatcttatcctctttaaacctcacagtttgaagatgcgactttgtttgattatactttttccaaaggagagaattaagagattatatttggaattacgcagacaaaatttgaaagacatcttataagtttgaaatccaattacaagtggaatttaaaattttcacgacttgtcaaccattgattctcaaataaagtgaaaaattattccaaaaacaaataaatattttttttatgaccaaatatgtcctcaagaacatatgaaagctctctagtcatgagtataaataacaagggctagctagctcttgtctactcataaaatatcatccatccatctcatgtaataaacaaaaattgagcttattaattataattgagaagaaaaaaaatcatgcctagagatcctttaatagtttctggagttgttggagatgttgttgatccattcacaagatgtgtagactttggtgtggtttacaacaatagggtggtctataatggatgttccttgaggccttcacaagttgtcaatcaacctagggttgacattgatggagacgatcttcgtactttttacactctggtataaactcatcgttttatttcatatgatatacatatatatatatatatatatatatatatatatatatattttctttctatttatacattttaatatctctaaattattaaccttttgtcaattgattatgagtagaagatcaaaaggacaatatgtgcaaaggcttctaattatgtgaatttgtgttagttttaattttgattcaccatctaagtacttgttttgtggtttttatttgaatttgagaactcataacatactatttatgataataaaaaatgttagtaacatgtatgtttaatattgcaagcttgaaaatatacaatatttttaaattactaataatgtcatgtaatacatttggatatacaatatggaaaattatttttcctaattttcaaaatatttgaaatgtttcttttctttttggaagattatggtggatcctgatgctccaaaccctagcaacccaaacctgagggaatatttgcactggtaagtcatctagcttatattatatatatatatatatatatatatatatattatataaatagataaaaatattcattttgttatatacttcttatttctcttaaatcaatcgtcgatagcgaagacaaaaatgtatgtgagattatataagaacctaaggaaagtattatttcataaaatgataactttctgatacacaaattaatcaatatttcaaataaataccaaatatcgaataacaacgtaaaaaaataataactattatcgattgcttaatccccttacaattaatgtacctaaacctctttttttttttaaaaaaaaaataataataataatgtttaacacattatttttttaataggttggtcacagatatcccagcagccacaggagcaacctttggtaagtttttcttacattattacctaatggctcgtaattacgcagtgacgaagcaagaaatttaaatatactttatatttacgatacattgtatccgtatcactacatttttaatataagacggttagtaatatacaaaatacaacttgtatcatcatcaccttagtagtacattattagtactataggcccaattatgactactaataaaataagacttaaaaagaaacataaaatcaaaatgaagtatatactatgtatataaatgtttttgaaacaaggaaaatacgcgtattgaatgtctttgttactaaactcaaactctcgttatacaggcaatgaagtcgtgggctacgagagcccacgaccctcaatgggaatccatcgttatattttcgtgttgtatcgacaattgggctgcgatgccatcgatgcaccggacataatcgattctagacaaaatttcaacacaagagactttgctaggtttcacaatctaggtttgcctgttgctgctgtttacttcaattgcaatagggaaggtggtaccggtggtcgtcgcctataaatcacccccctctcctcggggtgcgatccgttctcgaactctgtgtcaatgtcagatgttttgtgtaacggattttttgtttgatagtcactcagctaaattgcttattaactcagaaagtcatttttctttttatcgaagaaaattgaaattatgagataatagctattatagttgagtgataatctgaaaaaaaaaatcaaccataatatatattatatattatatatatacccaaaaaataaaagtct

SEQ ID NO: 8 - protein, 175 aa, Artificial Sequence
  Synthetic polynucleotide
MPRDPLIVSGVVGDVVDPFTRCVDFGVVYNNRVVYNGCSLRPSQVVNQPRVDIDGDDLRTFYTLIMVDPDAPNPSNPNLREYLHWLVTDIPAATGATFGNEVVGYESPRPSMGIHRYIFVLYRQLGCDAIDAPDIIDSRQNFNTRDFARFHNLGLPVAAVYFNCNREGGTGGRRL

SEQ ID NO: 9 - DNA, 110 bp, Artificial Sequence
  Synthetic polynucleotide
ggcagcagagagacctgaaaaagcaatttttctggaatttcagctgtttccaaactcaataagtatcttctagcaagagggaataggtgggaaaaaaaaaaagagatttc

SEQ ID NO: 10 - DNA, 497 bp, Artificial Sequence
  Synthetic polynucleotide
cgttgacttgcgcaagtcaacgttaacaccgttttcggggggtgacgatcgtcaccccaatgggtaagggttccatcgccgggaaacagctggagcttagtaatgaataaatgaactggactgactaatgtagtcagtccctcgatcgatgcatgcataccgtaaaggagaagctggtggaacgtgggatctctgtgcaaagctacaatggagatctattgtatgaaccgtgggaatatcggccggccacgcgacagtgcgctcgggtggtccatccactcgacacacccgccaccggcagccgctacccgcgcgcggccggtatattctggccttcccacaagggaaggccaggatcgtcgatgccgtggccaaggggtcattgagatagcctttcatgatttccgtctgcgcgcgcagacgggcaaggtagaagaccttggctgtaccattttttaacggaggagttactttctaagtatcatttagatttgaac

SEQ ID NO: 11 - DNA, 223 bp, Artificial Sequence
  Synthetic polynucleotide
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctacatttcaagatacc

SEQ ID NO: 12 - DNA, 223 bp, Artificial Sequence; identical to SEQ ID NO: 11
  Synthetic polynucleotide

SEQ ID NO: 13 - DNA, 255 bp, Artificial Sequence
  Synthetic polynucleotide
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctccattggataggaacagctatattgggtagttgtagcagttgtattcaaatgaatgcagcgttccgaaatatcatacctgccgatcaagaaacaaata

SEQ ID NO: 14 - DNA, 374 bp, Artificial Sequence
  Synthetic polynucleotide
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaacc

SEQ ID NO: 15 - DNA, 743 bp, Artificial Sequence
  Synthetic polynucleotide
ttaaaacagctctggggttgtacccaccccagaggcccacgtggcggctagtactccggtattgcggtacccttgtacgcctgttttatactcccttcccgtaacttagacgcacaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcatatg

SEQ ID NO: 16 - DNA, 730 bp, Artificial Sequence
  Synthetic polynucleotide
cacccactcacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttctttttatttttatattggctgcttatggtgacaattacagaattgttaccatatagctattggattggccatccggtgtgtaatagagctgttatatacctatttgttggctttgtaccactaactttaaaatctataactaccctcaactttgtattaactctcaatacagctgatcatg

SEQ ID NO: 17 - DNA, 744 bp, Artificial Sequence
  Synthetic polynucleotide
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcatatg